(19) United States
(12) Patent Application Publication
(10) Pub. No.: US 2024/0180843 A1
(43) Pub. Date: Jun. 6, 2024

(54) Thermally Stable Vaccine Formulations Utilising Metal Organic Framework (MOF) Shells

(71) Applicant: Commonwealth Scientific and Industrial Research Organisation, Acton,Australian Capital Territory (AU)

(72) Inventors: Ruhani Singh, Clayton, Victoria (AU); Daniel Layton, Clayton, Victoria (AU); Cara Doherty, Clayton, Victoria (AU)

(21) Appl. No.: 18/285,814

(22) PCT Filed: Apr. 27, 2022
(86) PCT No.: PCT/AU2022/050390

§ 371 Date: Oct. 5, 2023

(30) Foreign Application Priority Data
Apr. 27, 2021 (AU) 2021901241

(51) Int. Cl. A61K 9/51; A61K 39/00; A61K 39/12; A61K 47/46; C12N 7/00

(52) U.S. Cl. CPC A61K 9/5146; A61K 9/5192; A61K 39/12; A61K 47/46; C12N 7/00; A61K 2039/55505; A61K 2039/55555

(57) ABSTRACT

The present application relates to metal-organic framework (MOF) encapsulation or viral vaccines and vectors. The present application discloses methods for stabilizing viral vaccines and vectors and provides MOF encapsulated viral vaccines and vectors with improved stability.

can be dried in order to further increase their thermal stability. Another approach has been to engineer vaccines that can maintain their thermal stability without preservative adjuvants.

However, many of these thermally stabilized vaccines still have short shelf lives (<few hours to 7 days) when stored at elevated temperature (>37° C.). There is therefore a need for alternate thermal stabilisation approaches for vaccines including those vaccines where additives and stabilizers alone may not be sufficient.

### SUMMARY OF INVENTION

The present inventors applied metal-organic framework (MOF) mediated biomimetic mineralisation technique for live viral vaccine encapsulation and investigated the efficiency and efficacy of the resulting vaccine formulation thereof. And surprisingly demonstrated that MOF encapsulation of a live viral vaccine stabilises the vaccine for long term storage without freezing whilst retaining viral replication potential.

Accordingly, the present disclosure provides a stabilized composition comprising a live viral vaccine, a live-attenuated viral vaccine, an inactivated viral vaccine, a chimeric viral vaccine, a virus like particle vaccine, or a viral vector encapsulated within a Metal Organic Framework (MOF) shell. The vaccine or vector may be for example between about 20 nm and about 900 nm in size.

In one embodiment, the live-attenuated or inactivated viral vaccine is a whole pathogen live-attenuated or inactivated viral vaccine.

In one or a further embodiment, the stabilized composition comprises a vaccine or vector that is replication competent. In an alternate embodiment, the stabilized composition comprises a vaccine or vector that is replication incompetent, for example, replication-defective mutant viruses defective for one or more functions that are essential for viral genome replication or synthesis and assembly of viral particles.

In another embodiment, the present invention provides a stabilized composition comprising a live viral vaccine, a live-attenuated viral vaccine, an inactivated viral vaccine, a chimeric viral vaccine, a virus like particle vaccine, or a viral vector encapsulated within a Metal Organic Framework (MOF) shell, wherein the vaccine or vector is replication-competent in animal cells, for example, in mammalian cells or avian cells.

In another embodiment, the present disclosure provides a stabilized composition comprising a live viral vaccine, a live-attenuated viral vaccine, a chimeric viral vaccine, or a viral vector encapsulated within a Metal Organic Framework (MOF) shell, wherein the vaccine or vector is replication-competent in animal cells, for example, in mammalian cells or avian cells.

In another embodiment, the present invention provides a stabilized composition comprising a live viral vaccine, a live-attenuated viral vaccine, an inactivated viral vaccine, a chimeric viral vaccine, a virus like particle vaccine, or a viral vector encapsulated within a Metal Organic Framework (MOF) shell, wherein the vaccine or vector is an animal virus-based vaccine or viral vector that is replication-incompetent. In an embodiment, the animal virus based vaccine or viral vector is a replication defective adenovirus based virus or vector, for example, a chimpanzee adenovirus based virus or vaccine for use in mammals including humans, or a Fowlpox or turkey herpesvirus based virus or vector for use in avians. The animal virus based vaccine or viral vector may be used to deliver an antigenic payload (e.g., nucleic acid to generate protein for an immune response).

In another embodiment, the present disclosure provides a stabilized composition comprising a live viral vaccine, a live-attenuated viral vaccine, an inactivated viral vaccine, a chimeric viral vaccine, a virus like particle vaccine, or a viral vector encapsulated within a Metal Organic Framework (MOF) shell, wherein the vaccine or vector is not a plant based vaccine or vector, for example the virus or vector is not a chimeric potato virus X (PVX) or tobacco mosaic virus (TMV) based virus or vector.

Advantageously, the compositions of the disclosure maintain (at least partially) the physical stability and/or chemical stability and/or biological activity of the vaccine or vector. In one embodiment, the composition is characterized as having improved stability over 12 weeks as compared to a comparative composition comprising the vaccine or vector without the outer protective MOF shell. For example, the outer protective MOF shell reduces loss of virus titre over 12 weeks as compared to a composition comprising the vaccine or vector without the outer protective MOF shell. In some embodiments, the MOF protective shell composition comprises at least 1, or at least 2, or at least 3, or at least 4 log₁₀ more virus than the composition comprising the vaccine or vector without the outer protective MOF shell after 12 weeks of storage at temperatures up to 37°. In some embodiments, the composition may maintain at least 10%, at least 20%, at least 30%, at least 40%, least 50%, at least 55%, at least 60%, at least 70%, at least 75% of its activity after 12 weeks of storage at temperatures up to 37° C. In some embodiments the vaccine is stored between 4 to 37° C. In one embodiment, the vaccine is stored at 4° C. The structural integrity of the vaccine or vector may be determined by TEM imaging for example whilst its functional integrity may be demonstrated by retention of viral replication potential if it is a replication competent vaccine or vector, or its infectivity potential, or its ability to deliver antigenic payload (nucleic acid to generate protein for an immune response or directly deliver a protein payload for an immune response).

In one embodiment, the present disclosure provides a stabilized composition comprising a live viral vaccine, a live-attenuated viral vaccine, an inactivated viral vaccine, a chimeric viral vaccine, a virus like particle vaccine, or a viral vector encapsulated within a Metal Organic Framework (MOF) shell, wherein the stabilized composition maintains at least 5 log₁₀ of its viral replication potential or infectivity potential or its ability to deliver an antigenic payload after 12 weeks of storage at temperatures up to 37° C.

In another embodiment, the present disclosure provides a stabilized composition comprising a live viral vaccine, a live-attenuated viral vaccine, a chimeric viral vaccine, or a viral vector encapsulated within a Metal Organic Framework (MOF) shell, wherein the stabilized composition maintains at least 5 log₁₀ of its viral replication potential after 12 weeks of storage at temperatures up to 37° C. In some embodiments, the vaccine or vector is replication-competent in animal cells, for example, in mammalian cells or avian cells.

In another embodiment, the present disclosure provides a stabilized composition comprising a live viral vaccine, a live-attenuated viral vaccine, an inactivated viral vaccine, a chimeric viral vaccine, a virus like particle vaccine, or a viral vector encapsulated within a Metal Organic Framework (MOF) shell, wherein the stabilized composition comprises at least 1 log₁₀ more virus than the composition comprising the vaccine or vector without the outer protective MOF shell after 12 weeks of storage at temperatures up to 37°. In some embodiments, the vaccine or vector is an animal virus-based vaccine or viral vector that is replication-incompetent. In an embodiment, the animal virus based vaccine or viral vector is a replication defective adenovirus based virus or vector.

In one embodiment, the present disclosure provides a stabilized composition comprising a live viral vaccine, a live-attenuated viral vaccine, an inactivated viral vaccine, a chimeric viral vaccine, a virus like particle vaccine, or a viral vector encapsulated within a Metal Organic Framework (MOF) shell, wherein the stabilized composition maintains at least 5 log₁₀ of its infectivity potential after 12 weeks of storage at temperatures up to 37° C.

In another embodiment, the present disclosure provides a stabilized composition comprising a live viral vaccine, a live-attenuated viral vaccine, a chimeric viral vaccine, or a viral vector encapsulated within a Metal Organic Framework (MOF) shell, wherein the stabilized composition maintains at least 5 log₁₀ of its infectivity potential after 12 weeks of storage at temperatures up to 37° C.

In one embodiment, the present disclosure provides a stabilized composition comprising a live viral vaccine, a live-attenuated viral vaccine, an inactivated viral vaccine, a chimeric viral vaccine, a virus like particle vaccine, or a viral vector encapsulated within a Metal Organic Framework (MOF) shell, wherein the stabilized composition maintains at least 5 log₁₀ of its ability to deliver an antigenic payload after 12 weeks of storage at temperatures up to 37° C.

In another embodiment, the present disclosure provides a stabilized composition comprising a live viral vaccine, a live-attenuated viral vaccine, a chimeric viral vaccine, or a viral vector encapsulated within a Metal Organic Framework (MOF) shell, wherein the stabilized composition maintains at least 5 log₁₀ of its ability to deliver an antigenic payload potential after 12 weeks of storage at temperatures up to 37° C.

In some embodiments, the MOF is based on a carboxylic acid based precursor linker (e.g., a lactic acid based MOF), a dicarboxylate acid based linker (e.g., fumaric acid, succinic acid, or malic acid based MOF), a tricarboxylate linker (e.g. benzene tricarboxylate based MOF), an imidazolate linker (ZIFs) and other organic molecules where for example, peptides or small molecules may be used to make the MOF biocompatible.

In one embodiment, the MOF is a zeolitic imidazolate framework (ZIF), for example, ZIF-8, ZIF-10, ZIF-90. or ZIF-L. In one embodiment the ZIF is ZIF-8. In another embodiment, the MOF is an aluminium based MOF, for example, aluminium fumarate, aluminium-cyclodextrin (alpha, beta, gamma), aluminium-tartrate, aluminium-gallate, MIL-53(AI), MIL-118A(AI), CAU-23(AI), MIL-96, MIL-100, MIL-101, NH-MIL-101, MIL-101—NH2, MIL-68, CAU-1, CAU-1-OH, CAU-10, CAU-23, MIL-69, MIL-116, MIL-118, MIL-120, MIL-121, MIL-122 and MIL-160. CAU-3, CAU-4, CAU-6, CAU-8, CAU-11, CAU-12, CAU-13, and CAU-15. BIT-72, BIT-73, BIT-74, KMF-1, PCN-333, MOF-253, DUT-5, 467-MOF, or AL-MIL-53—NH2. In one embodiment, the MOF is aluminium fumarate. The MOF may be crystalline, amorphous, or mixed phase.

In one embodiment, the composition is dried, for example, freeze dried. In one or a further embodiment, the composition comprises one or more excipients, for example, trehalose, or skim milk, or a combination thereof. The present disclosure also provides a method for producing a stabilized composition, the method comprising:
a. providing a live viral vaccine, a live-attenuated viral vaccine, an inactivated viral vaccine, a chimeric viral vaccine, a virus like particle vaccine, or a viral vector;
b. providing a ligand precursor;
c. providing a metal salt;
d. reacting the vaccine or vector, the ligand precursor and the metal salt to form a metal organic framework shell encapsulating the vaccine or vector.

Advantageously, the present disclosure provides a universal approach for the thermal stabilisation of vaccines and vectors based on MOF material encapsulation.

In one embodiment, one or more of the vaccine or vector, the ligand precursor and the metal salt are provided in solution in one or mixed solvents, for example, water, alcohol, or other organic solvent.

In one or a further embodiment, the solution comprises one or more excipients.

In one or a further embodiment, the ligand precursor is 2-methylimidazole, for example, 80 to 640 mM 2-methylimidazole in water. In another embodiment, the ligand precursor is sodium aluminate, for example, 5 to 45 mM sodium aluminate in water.

In one or a further embodiment, the metal salt is zinc acetate, for example, 20 to 160 mM zinc acetate dihydrate in water. In another embodiment, the metal salt is fumaric acid, for example, 5 to 45 mM fumaric acid in water.

In one embodiment, the metal salt and ligand precursor is provided at a ratio from 100:1 to 1:100. For example, where the metal salt is zinc acetate and the ligand precursor is 2-methylimidazole, the metal salt:ligand precursor ratio is preferably between 1:4 and 1:8. In another example, the metal salt is fumaric acid and the ligand precursor is sodium aluminate and the metal salt:ligand precursor ratio is 1:1.

In one or a further embodiment, the vaccine or vector, the ligand precursor and the metal salt solution are incubated for about 5 to 30 minutes.

In one or a further embodiment, the method further comprises centrifuging the reaction mixture of step (d) to pellet the metal organic framework encapsulating the vaccine or vector.

In one or a further embodiment, the method further comprises adding one or more excipients, for example, trehalose (for example, between 0.5-20% w/w solution such as 5-10% w/w solution), before the metal organic framework shell forms.

In one or a further embodiment, the pellet is collected.

In one or a further embodiment, the pellet is dried, for example, freeze dried. In one embodiment, the method further comprises adding one or more excipients, for example, skim milk (or example, between 0.5-20% w/w solution such as 5-10% w/w solution), prior to drying.

The stabilized vaccine or vector composition may be administered to a subject as part of a vaccination strategy. In one embodiment the subject is a human. In another embodiment, the subject is an avian, for example, a chicken. The stabilized vector may also be administered to a subject for gene therapy or drug therapy purposes. In one embodiment, the composition is administered as is (i.e., with the outer protective MOF shell). In an alternate embodiment, the vaccine or vector is first release from the MOF shell.

Accordingly, the present disclosure also provides a method of preparation of the composition of the disclosure for administration, wherein the method comprises adding a release buffer, for example citrate buffer or an EDTA buffer, to the composition to chelate the metal ions causing MOF disintegration, and thereby release the vaccine or vector. In one embodiment, between about 10 mM to about 500 mM sodium citrate at a pH of about 5.0 is used, for example, 50 mM sodium citrate.

The present disclosure is not to be limited in scope by the specific examples described herein, which are intended for the purpose of exemplification only. Functionally equivalent formulations, compositions and methods are clearly within the scope of the present disclosure.

Any example/embodiment of the present disclosure herein shall be taken to apply mutatis mutandis to any other example/embodiment of the disclosure unless specifically stated otherwise.

### DESCRIPTION OF THE DRAWINGS

FIG. 1. Synthesis and characterisation of ZIF-8@NDV prepared using biomimetic mineralisation technique. Schematic (a) illustrates the proposed mechanism; the presence of viral capsid proteins increases the local concentration of MOF precursors, facilitating the formation of MOF prenucleation clusters, white (step 1). This leads to biomimetic growth of MOF crystals around the virus (step 2); (b) The in vitro assessment of median tissue culture infectious dose (TCID50/mL) shows good encapsulation efficiency in ZIF-8 forming ZIF-8@NDV with a comparable titre to the control NDV vaccine stored at −80° C. The PXRD spectra in (c) show similarity between the ZIF-8@NDV (50% ethanol processed: crystalline), and the control ZIF-8 MOF. However, an aqueous synthesis rendered the ZIF-8@NDV (aqueous synthesis; amorphous) with an amorphous phase. NDV spectra shows peaks for NaCl and KCl salts from virus preparation medium, which are absent in the MOF composites. All data is presented as mean±SD (n=3). Statistical significance was tested against the control (NDV) group (one-way ANOVA p<0.05 with Dunett's multiple comparison test; ****, p<0.0001).

FIG. 2. (i) The effect of biomimetic mineralization and post-processing (aqueous or ethanol) on NDV virions & (ii, iii) Investigation of MOF growth around NDV virions using surface charge measurement.

FIG. 3. (a, b) ZIF-8@WSN and Alfum@WSN—Encapsulation efficiency and Infective titre quantification and (c, d) Powder X-ray diffraction spectrum for ZIF-8@WSN and Alfum@WSN.

FIG. 4. The effect of biomimetic mineralization and post-processing (aqueous or ethanol) on WSN virions. (a) Dynamic light scattering (DLS) analysis of the virus particles or virion size before encapsulation and after their release from the ZIF-8@WSN and Alfum@WSN indicate structural integrity is maintained during the encapsulation and release process.

(b) Zeta potential plot of pristine WSN virus followed by virus with addition of first precursor (2-methylimidazole—Hmlm) and second precursor (Zinc Acetate ZnAc) and the final ZIF-8@WSN composites after the synthesis and washing steps. Y-axis units expressed as millivolt.

(c) Zeta potential values as plotted in (b); error=standard deviation Std. (n=3). The surface charge analysis shows the initial negative surface charge of the virions, WSN which is slightly enhanced on addition of the imidazole precursor. The addition of the zinc salt inverts the zeta potential to nearly (+)33 mV. The present inventors postulate this is due to the presence of excess precursors as the final composite post-washing steps have a zeta potential of about (+)7 mV.

FIG. 5. Scanning electron microscopy (SEM) and transmission electron microscopy (TEM) images for NDV (i, ii), and ZIF-8@NDV (iii, iv) (scale—200 nm).

FIG. 6. TEM and SEM micrographs for the Influenza A WSN virus, ZIF-8@WSN and Alfum@WSN.

FIG. 7. Transmission Electron Microscopy visualization of ZIF-8 encapsulation and release of the NDV live viral vaccine. Schematic Steps 1 to 4 show the sequence of events captured using TEM images (a) to (d). The NDV virus (1, a) is encapsulated using the ZIF-8 MOF forming an amorphous composite (2, b). The electron density image of the composite correlates with its SEM image in inset b. A sodium citrate buffer (pH 5.0, 50 mM) was used to chelate the zinc ions causing MOF disintegration (3, c) releasing structurally intact NDV virions (4, d).

FIG. 8. (i) Transmission Electron Microscopy visualization of ZIF-8 encapsulation and release of the Influenza A WSN virus and (ii) Transmission Electron Microscopy visualization of Alfum encapsulation and release of the Influenza A WSN virus.

FIG. 9. Energy-dispersive x-ray spectroscopy (EDS) of ZIF-8@NDV, ZIF-8@WSN, and Alfum@WSN.

FIG. 10. Storage stability of the ZIF-8@NDV formulation in solution or wet-pellet form.

FIG. 11. The Effect of Freeze-drying and Synthesis of stable freeze-dried MOF@Vaccine composite. (a) Subjecting the NDV and ZIF-8@NDV to freeze-drying (FD) results in significant loss of virus titre due to the FD stress. (b) To synthesize a stable composite, the addition of different combinations of trehalose (T) and skim milk (SM) were investigated. The addition of T to stabilise the viral particles before MOF encapsulation, followed by addition of SM before FD in ZIF-8@NDV+T/SM significantly protected the viral titre. (all data is presented as means±SD, NDV and NDV FD; NDV FD and NDV+T/SM; ZIF-8@NDV and ZIF-8@NDV FD were compared using unpaired t-tests (p<0.05). For all other data—one-way ANOVA p<0.05 with Dunett's multiple comparison test; *, p<0.05; **, p<0.01; ****, p<0.0001).

FIG. 12. ZIF-8@NDV+T/SM PXRD and EDS characterisation.

FIG. 13. Storage stability of NDV, NDV+T/SM and ZIF-8@NDV+T/SM at (a) 4° C., (b) room temperature (R.T.) and (c) 37° C. over a period of 12 weeks. (d) shows TEM micrographs of NDV virions released from the NDV, NDV+T/SM and ZIF-8@NDV+T/SM composites after their storage at 4° C., RT, and 37° C. over a period of 12 wks. The structure of NDV virions is revealed by negative contrast using phosphotungstic acid stain on the TEM samples. (two representational images from each sample, scale—200 nm). Statistical significance was tested between the three formulation types at each temperature and each time point using one-way ANOVA p<0.05 with Tukey's multiple comparison test (*, p<0.05; **, p<0.01;***, p<0.001****, p<0.0001) All data from (a-b) are presented as mean±SD.

FIG. 14. Optimisation of ZIF-8@NDV synthesis conditions.

FIG. 15. Small angle X-ray spectroscopy (SAXS) analysis of ZIF-8@NDV.

FIG. 16. Encapsulation efficiency and infectivity of ZIF-8@WSN and Alfum@WSN.

FIG. 17. Thermogravimetric analysis of the Control ZIF-8, ZIF-8@NDV and ZIF-8@NDV(E) lower thermal stability of the composite material consistent with the presence of NDV. Further, the addition of Trehalose and Skim Milk led to a further drop in material thermal stability for the ZIF-8@NDV+T/SM composite.

FIG. 18. The initial titre for the three formulations, NDV, NDV+T/SM and ZIF-8@NDV+T/SM.

FIG. 19. Immunofluorescence images from infected DF1 cells infected with a 100× dilution of the original viral titre in form of ZIF-8@NDV+T/SM, NDV+T/SM and NDV that were stored at room temperature for a period of 12 weeks (scale bar—400 μm).

FIG. 20. SEM micrographs of ZIF-8@NDV+T/SM Control (t=0) (a) and the composite after 12 weeks (t=12 wk) of storage at 4° ° C.(b), RT(c), and 37° C. (d) show no distinctive change in MOF composite morphology (50,000× magnification: scale −200 nm).

FIG. 21. PXRD spectra for ZIF-8@NDV+T/SM composites after their storage at 4° C., RT, and 37° C. over a period of 12 wks.

### DETAILED DESCRIPTION

Before describing the present invention in detail, it is to be understood that the terminology used herein is for the purpose of describing particular embodiments only and is not intended to be limiting.

As used in this specification and the appended claims, the singular forms “a”, “an” and “the” include plural referents unless the content clearly dictates otherwise. Thus, for example, reference to an antigen, a metal salt, a ligand precursor includes a combination of two or more such molecules.

Throughout the description and claims of the specification, the word “comprise” and variations of the word, such as “comprising” and “comprises”, is not intended to exclude other additives, components, integers or steps.

The term “about”, as used herein, indicates the value of a given quantity can include quantities ranging within 10% of the stated value, or optionally within 5% of the value, or in some embodiments within 1% of the value.

Unless specifically defined otherwise, all technical and scientific terms used herein shall be taken to have the same meaning as commonly understood by one of ordinary skill in the art

All publications or patents cited herein are entirely incorporated herein by reference. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, N.Y. (1987 including all updates until present); Sambrook, et al., Molecular Cloning: A Laboratory Manual, 2nd Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994 including all updates until present); Colligan et al., Current Protocols in Protein Science, John Wiley & Sons, NY, N.Y., (1997 including all updates until present).

A reference herein to a patent document or other matter which is given as prior art is not to be taken as admission that the document or matter was known or that the information it contains was part of the common general knowledge as at the priority date of any of the claims.

As used herein “Metal-Organic Frameworks (MOFs)” are one-two or three dimensional organic-inorganic hybrid coordination networks composed of metal ions or clusters (termed secondary binding units (SBUs) bridged by organic ligands. The organic ligands may be carboxylates, or anions, such as phosphonate, sulfonate, and heterocyclic compounds. The geometry is determined by the coordination number, coordination geometry of the metal ions, and the nature of the functional groups. A variety of SBU geometries with different number of points of extension such as octahedron (six points), trigonal prism (six points), square paddle-wheel (four points), and triangle (three points) have been observed in MOF structures. The final framework topology of MOF is governed by both SBU connectors and organic ligand linkers. Depending upon the nature of the system used, infinite-extended polymeric or discrete-closed oligomeric structures can arise. MOFs may have pore openings up to 2 nm size (microporous) or may have a pore size of 2-50 nm (mesoporous). The synthesis of MOFs involves reaction conditions and simple methods such as solvothermal, ionothermal, diffusion, microwave methods, ultrasound-assisted, template-directed syntheses, and others. Various MOFs composed of different metal ions and organic ligands have been described. Particularly useful MOFs in the stabilisation of vaccines of the disclosure are biocompatible. Such MOFs may be synthesized from non-toxic cations such as calcium, iron, zinc, aluminium, molybdenum, sodium, copper, potassium and magnesium. The MOF may be amorphous, or crystalline, or a mixed phase.

As used herein, “MOF shell” refers to a MOF layer that encapsulates the vaccine or vector to form a protective coating for the storage of vaccine or vector. The vaccine or vector does not localise within the MOF pores or between MOF layers owing to its size. As used herein, “zeolitic imidazolate framework” (or “ZIF”) refer to microporous structures having frameworks commonly found in zeolites and/or in other crystalline materials wherein each vertex of the framework structure is comprised of a single metal ion and each pair of connected adjacent vertices of the framework structure is linked by nitrogen atoms of an imidazolate anion or its derivative as the ligand. ZIFs are a subset of MOFs. The frameworks can comprise any of the networks defined in the Atlas of Zeolite Structure Types and the Reticular Chemistry Structure Resource (RCSR) Database known in the literature. Particularly useful ZIFs in the stabilisation of vaccines of the disclosure are biocompatible. Such ZIFs may be synthesized from non-toxic cations such as calcium, iron, zinc, aluminium, molybdenum, sodium, copper, potassium and magnesium. The ZIF may be amorphous, or crystalline, or a mixed phase. In one embodiment, the ZIF is amorphous.

A “stable” formulation or composition is one in which the vaccine or vector therein essentially maintains its physical stability (or structural integrity) and/or chemical stability and/or biological activity upon storage and on release from the MOF shell. For example, the vaccine or vector maintains its infectivity (i.e., can bind and enter a cell) and/or its replication capacity (in the case of replication competent vaccines or vectors) and/or its ability to deliver payload (e.g., nucleic acid to generate protein for an immune response or directly deliver a protein payload for an immune response). As used herein “maintains” will be understood to include partial retention, for example, at least 10%, at least 20%, at least 30%, at least 40%, at least 50%, at least 60%, at least 70%, at least 75%, at least 80%, at least 85%, at least 90%, at least 95% of its activity prior to encapsulation within a MOF shell. Stability can be measured at a selected temperature for a selected period. Trend analysis can be used to estimate an expected shelf life before a material has actually been in storage for that time period.

As used herein, “antigen” refers to a biomolecule capable of eliciting an immune response in a host. In some embodiments, the antigen elicits a protective immune response. The antigen may elicit a humoral or cellular immune response, or both.

As used herein, the terms “subject” and “host” are intended to include living organisms such as mammals. Examples of subjects or hosts include, but are not limited to, horses, cows, sheep, pigs, goats, dogs, cats, rabbits, guinea pigs, rats, mice, gerbils, non-human primates, humans and the like, non-mammals, including, for example, non-mammalian vertebrates, such as birds (e.g., chickens or ducks) fish or frogs (e.g., Xenopus), non-mammalian invertebrates, as well as transgenic species thereof.

As used herein, the term “immune response” is intended to include, but is not limited to, T and/or B cell responses, that is, cellular and/or humoral immune responses. In one embodiment, the claimed methods can be used to stimulate cytotoxic T cell responses. The claimed methods can be used to stimulate both primary and secondary immune responses. The immune response of a subject can be determined by, for example, assaying antibody production, immune cell proliferation, the release of cytokines, the expression of cell surface markers, cytotoxicity, and the like.

As used herein, the term “adjuvant” includes, but is not limited to, agents which potentiate the immune response to an antigen.

As used herein, the term “ambient room temperature” refers to typical controlled indoor temperatures, such as from about 16° C. to about 27° C., or more typically from about 18° C. to about 25° C., and often about 24° C.

As used herein, the term “dry” or “dried” in reference to the solid vaccine formulations described herein refers to a composition from which a substantial portion of any water has been removed to produce a solid phase of the composition. The term does not require the complete absence of moisture. The vaccine compositions described herein generally have a moisture content from about 0.1% by weight and about 25% by weight.

### Vaccine Compositions

The vaccine compositions may comprise a live viral vaccine (e.g., whole virus or whole live attenuated virus vaccine), an inactivated viral vaccine, a chimeric viral vaccine (e.g., live attenuated), a virus like particle vaccine, a viral vector (e.g., replicating viral vector), or a combination thereof. The vaccine or vector may be replication competent or incompetent. The vaccine or vector comprises one or more antigens.

Viruses include DNA or RNA viruses. As used herein, RNA viruses include, but are not limited to, virus families such as picoruaviridae (e.g., polioviruses), reoviridae (e.g., rotaviruses), logaviriclae (e.g., encephalitis viruses, yellow fever virus, rubella virus), orthomyxoviridae (e.g., influenza viruses), paramyxoviridae (e.g., respiratory syncytial virus (RSV), measles virus (MV), mumps virus (MuV), parainfluenza virus (PIV)), rhabdoviridae (e.g., rabies virus (RV)), coronaviridae, bunyaviridae, flaviviridae (e.g., hepatitis C virus (HCV)), filoviridae, arenaviridae, bunyaviridae, and retroviridae (e.g., human T-cell lymphotropic viruses (HTLV), human immunodeficiency viruses (HIV)). As used herein, DNA viruses include, but are not limited to, virus families such as papovaviridae (e.g., papilloma viruses), adenoviridae (e.g., adenovirus), herpesviridae (e.g., herpes simplex viruses, e.g., HSV-1, HSV-2; varicella zoster virus (VZV); Epstein-Barr virus (EBV); cytomegalovirus (CMV); human herpesviruses, e.g., HHV-6 and HHV-7; Kaposi's sarcoma-associated herpesvirus (KSHV) and the like), and poxviridae (e.g., variola viruses). These and other viruses and viral proteins are included in the present invention and are described further in Knipe et al. Field's Virology, 4th ed., Lippincott Williams & Wilkins, 2001, incorporated herein by reference in its entirety for all purposes.

In some embodiments, the vaccine compositions comprise whole disease causing virus.

In other or further embodiments, the vaccine compositions comprise live attenuated virus. Such live attenuated viral vaccines are derived from disease-causing viruses that have been ‘weakened’ so that they elicit an immune response, preferably a protective immune response, but do not cause disease or only very mild disease in vaccinated subjects. This “weakening” may be achieved through genetic modification of the virus either as a naturally occurring phenomenon or by recombinant means.

In other or further embodiments, the vaccine compositions comprise a chimeric virus. A chimeric virus comprises nucleic acid fragments or proteins from two or more different viruses. In some embodiments, these chimeric viruses are live attenuated.

In other or further embodiments, the vaccine compositions comprise inactivated viruses. Such inactivated viral vaccines typically comprise whole viruses that have been killed through physical or chemical processes. These killed organisms cannot cause disease.

In other or further embodiments, the vaccine compositions comprise virus-like particles (VLPs). VLPs are molecules that closely resemble viruses but are non-infectious because they contain no viral genetic material. They can be naturally occurring or synthesized through the individual expression of viral structural proteins, which can then self-assemble into the virus-like structure. In some embodiments, the one or more antigens in a VLP vaccine are the viral structural proteins themselves. Alternatively, the VLPs can be manufactured to present antigens from another pathogen on the surface, or even multiple pathogens at once.

In other or further embodiments, the vaccine compositions comprise viral vectors. Such viral vector vaccines use ‘harmless’ or attenuated viruses to deliver the genetic code of one or more target vaccine antigens to cells of the body, so that they can produce protein antigen(s) to stimulate an immune response. Viral vectored vaccines can be grown in cell lines using techniques well known in the art. In one embodiment, the viral vectors are replicating viral vectors, that is, they retain the ability to make new viral particles alongside delivering the vaccine antigen(s) when used as a vaccine delivery platform. As with live attenuated whole pathogen vaccines this has the inherent advantage as a replicating virus that it can provide a continuous source of vaccine antigen over an extended period of time compared to non-replicating vaccines, and so is likely to produce a stronger immune response. Replicating viral vectors are typically selected so that the viruses themselves cannot cause disease. The skilled person will appreciate that stabilised vector compositions of the disclosure may be used to deliver not only vaccine antigen(s) but can be used for gene therapy and drug delivery.

Viral vectors for which both replicating and non-replicating forms are available include adenoviruses and poxviruses. Vectors designed primarily as replication-defective include adeno-associated virus, alphavirus, and herpesvirus, while replicating vectors include measles virus and vesicular stomatitis virus.

In one embodiment the vector is an adenoviral vector. Advantageously, adenoviral vectors can infect both dividing and non-dividing cells, express high levels of transgene, can grow to high titers in vitro, do not integrate in the host genome, and/or are physically and genetically stable. Adenoviral vectors can infect dendritic cells, upregulate co-stimulatory molecules, and elicit cytokine and chemokine responses, thus effectively presenting antigens to the immune system and eliciting potent immune responses. As adenoviruses target epithelial cells, they are prime candidates for elicitation of mucosal as well as systemic immunity.

Adenovirus (Ad) can be rendered replication defective by deletion of the E1 region genes, essential for replication. Such vectors generally have the non-essential E3 region deleted as well, in order to create more space for foreign genes. An expression cassette is then inserted with the transgene under the control of an exogenous promoter. In one embodiment, the adenoviral vector is a non-replicating Ad5 vector.

In some embodiments, a non-human adenovirus of chimpanzee origin is used, or an engineered chimeric vector in which the hypervariable region(s) of the hexon protein of Ad5, for example, targeted by Ad neutralizing antibodies, are replaced with corresponding regions of a rare Ad serotype such as Ad48. In some embodiments, different adenoviral vectors are used in prime/boost regimens (for the prime and boost immunizations) to focus the immune response on the inserted gene while avoiding anti-vector immunity induced by prior immunizations.

In some embodiments, the adenoviral vector is a replication-competent adenoviral vectors. Such vectors typically have the E3 region deleted, and as a result have a more limited clone capacity of 3-4 kb compared to replication-defective adenovirus. The vectors possess other advantages, however, that offset this limitation. One of the most practical is their ‘dose-sparing’ effect. The estimated dosages of replicating adenovirus-recombinants, based on the safe doses of licensed, oral wild-type Ad4 and Ad7 vaccines, are at least 2-3 logs lower than those of non-replicating Ad5 recombinants.

This dose-sparing effect, attributable to the subsequent replication of the vaccine vector in vivo, offers a powerful practical advantage for future manufacturers of the vaccine who would need to produce sufficient material for worldwide use.

The main scientific advantage of replicating adenovirus-recombinants is their mimicking of a natural adenoviral infection, resulting in induction of cytokines and co-stimulatory molecules that provide a potent adjuvant effect. Overall, the replicating vector can provide a complete immune response, including elements of innate immunity, an important component of a rapid response to an invading organism, as well as humoral, cellular, and mucosal immune responses.

In another embodiment, the vector is an adeno-associated virus (AAV). Adeno-associated virus (AAV) is a small single-stranded, non-pathogenic DNA virus containing only two genes that can be replaced with foreign genes. This leaves only the terminal ITRs to allow high level expression of the inserts. The vector infects muscle cells and can provide long lasting expression from either episomal or integrated genomic forms. As a non-enveloped vector, AAV exhibits physical stability; in particular its resistance to acid suggests a potential use in oral delivery.

In another embodiment, the vector is an alphavirus such as for example, a Venezuelan equine encephalitis virus (VEE), Sindbis virus (SIN), Semliki forest virus (SFV), or VEE-SIN chimera. Alphaviruses are single-stranded positive-sense RNA viruses that replicate in the cytoplasm of infected cells, and therefore have no potential for integrating into the host genome. Generally, to circumvent safety concerns, alphavirus vectors are engineered as non-replicating replicon particles in which structural gene products are deleted to accommodate a foreign gene of up to 5 kb, while structural proteins are provided in trans from two helper transcripts that lack a packaging signal. Deletion of the structural genes provides a further advantage in reducing immunity to the vector and enabling sequential immunizations. Advantageously, the vector is naturally targeted to dendritic cells in draining lymph nodes, where the transgene is typically expressed at high levels, leading to good immune responses. Immunogenicity may be further enhanced as the self-amplification of the vector RNA occurs through double-stranded RNA intermediates that stimulate activation of the interferon cascade, mimicking innate immunity. The vector also typically induces apoptosis in some cell types, thereby leading to cross-priming. Alphavirus vectors can be engineered to secrete proteins encoded by the transgenes, and additionally, can be designed to express heterologous proteins on the surface of infectious virus particles.

In another embodiment, the vector is an herpesvirus vector. Such vectors have been used most extensively in gene therapy applications related to the central or peripheral nervous system. The large enveloped double-stranded DNA viruses not only infect a variety of tissue types but also target mucosal surfaces and therefore are advantageous for elicitation of mucosal immune responses. The vectors can accommodate large foreign gene inserts and are biased for induction of Th1 cellular responses.

Additionally, HSV-1 activates TLR2 for induction of pro-inflammatory cytokines and TLR9 for induction of type I interferons. Both replication-competent and incompetent vectors have been developed. While replication-competent herpesvirus vectors are advantageous in many applications for their persistence, replication-deficient hervesvirus vaccine vectors also induce durable immune responses.

In another embodiment, the vector is a poxvirus vector. In addition to adenovirus vectors, poxvirus vectors are among the most heavily exploited for vaccine development. Non-replicating poxvirus vectors include modified vaccinia virus Ankara (MVA), replication deficient due to loss of approximately 15% of its genome upon repetitive serial passaging in chick embryo fibroblasts; NYVAC, derived from the Copenhagen strain of vaccinia and rendered replication incompetent by 18 specific engineered deletions; and avipox vectors: canarypox (ALVAC) and fowlpox (FPV). The latter, naturally restricted to growth in avian cells, can infect mammalian cells but do not replicate. Mammalian poxviruses have a double-stranded DNA genome of approximately 130 kb and avian poxviruses of about 300 kb. These large genomes allow the insertion of more than 10 kb of foreign DNA. Further, gene products are expressed at high levels, in general resulting in potent cellular immune responses.

In another embodiment, the vector is a vesicular stomatitis virus (VSV). VSV vectors are a comparatively new addition to the group of replication-competent viral vaccine vectors, as knowledge of how to manipulate the negative, single-stranded RNA genome was only relatively recently acquired. Advantages of the vector include its replication in the cytoplasm, thus avoiding integration into host DNA, a high level of transgene expression due to shutting down host mRNA translation, ease of production due to a rapid life cycle, limited pre-existing immunity in the population, and ability to be administered mucosally. The natural hosts for VSV infection are insects and livestock. In rare cases where the virus has been transmitted to humans, it has been asymptomatic, or caused only mild symptoms. Nevertheless, as a replicating vector, it has been vigorously investigated for safety. VSV has been found to be neurovirulent in rodents and also non-human primates following direct intracranial inoculation. The immunogenicity of attenuated vectors may be increased by increasing transgene expression by shifting the position of the transgene from the 5′end of the genome to the 3′ end, co-expressing immune modulators, targeting of dendritic cells, and combination strategies with other vector delivery systems.

The skilled person will appreciate that the choice of vector will be determined by the specific vaccine application. One consideration is choosing a vector is whether it will be used in a prophylactic or therapeutic application. In people already infected with an infectious agent, the benefit of a therapeutic vaccine may outweigh some risk attributed to the vector itself. By contrast, prophylactic vaccines are intended for healthy people, not only adults but also children and infants. Therefore, safety is of importance.

Vector selection also requires an understanding of the biology of the infectious agent for which the vaccine is being developed and knowledge of the course of the resultant disease. The mode of transmission of the infectious agent will impact vector choice. Moreover, natural recovery from disease will often highlight immune responses correlated with control or eradication of the infectious agent, providing information with regard to the type of immune response desired: cellular, mucosal, and/or humoral.

With regard to anti-vector immunity, an initial definition of the target population to be vaccinated is preferable in selecting a vector. Adult vaccines may already be heavily exposed to a particular viral vector and therefore exhibit high levels of anti-vector immunity. Infants may have acquired maternal antibodies to potential vaccine vectors, precluding effective vaccination.

Practical features are as important as the scientific ones. The capacity of the viral vector for foreign DNA must be sufficient for the gene(s) to be inserted. If more than one gene product needs to be expressed, a vector with a large capacity would be advantageous, rather than the use of multiple recombinants. A manufacturing strategy able to provide vaccine for use in millions of people worldwide is also an important consideration. A system for large scale production must be available, and the viral recombinant must be genetically stable in order to maintain its integrity through multiple passages in order to reach desired quantities of vaccine material.

The vaccine compositions may be monovalent comprising a single strain of a single antigen or polyvalent comprising two or more strains/serotypes of the same antigen. The vaccine compositions may comprise two or more antigens to prevent different disease or to protect against multiple strains causing the same disease. Such combination vaccines can be useful in overcoming logistic constraints of multiple injections.

As used herein, “biologically effective amount” refers to the amount of the one or more antigens needed to stimulate or initiate the desired immunologic response (e.g., protective immune response to the vaccine). Thus, the amount of the one or more antigens needed to achieve the desired immunological response will necessarily vary depending on a variety of factors including the type of antigen, the site of delivery (e.g., subcutaneous or intramuscular), and the dissolution and release kinetics for delivery of the antigen.

In some embodiments, the vaccine compositions further include one or more excipients including stabilizers, adjuvants, antibiotics, and preservatives.

Stabilizers can be used to help the vaccine maintain its effectiveness during storage. Vaccine stability is essential, particularly where the cold chain is unreliable. Instability can cause loss of antigenicity and decreased infectivity of live attenuated viruses. Factors affecting stability are temperature and acidity or alkalinity of the vaccine (pH). Stabilizing agents include MgCl₂, MgSO₄, lactose-sorbitol and sorbitol-gelatine.

Embodiments of the present application include vaccine compositions comprising one or more antigens and one or more selected excipients in a dry solid formulation. The one or more selected excipients may advantageously improve the stability of the one or more antigens during drying and storage of the vaccine compositions. Such stabilizers include, for example, one or more amino acids or one or more carbohydrates, or combinations thereof. For example, one or more amino acids selected from the group consisting of serine, asparagine, glycine, threonine, histidine, proline, taurine, and combinations thereof, and/or one or more carbohydrates selected from the group consisting of sucrose, trehalose, sorbitol, maltose, ducitol, and combinations thereof.

The one or more excipients may be present in the vaccine composition in a total amount from about 1% to about 90% by weight. For example, the one or more excipients may be present in the composition in a total amount from about 2 to about 75%, from about 5% to about 50%, or from about 5% to about 20%. In some embodiments, two excipients, for example, an amino acid and a carbohydrate, are present in the vaccine composition at a ratio from about 1:15 to about 15:1. For example, the excipients may be present in the composition at a ratio of about 1:9 to about 9:1, about 1:2, or about 1:1.

Adjuvants can be added to vaccine compositions of the disclosure to stimulate the production of antibodies against the vaccine to make it more effective. Adjuvants have been used to improve the immune response to vaccine antigens, most often in inactivated (killed) vaccines. The skilled person would appreciate that several different types of adjuvants could be used in the vaccine compositions of the disclosure.

Antibiotics (in trace amounts) may be used during the manufacturing phase to prevent bacterial contamination of the tissue culture cells in which the viruses are grown. Usually only trace amounts appear in vaccines.

Preservatives can be added to multidose vaccines to prevent bacterial and fungal growth. They include a variety of substances, for example, Thiomersal, Formaldehyde, or Phenol derivatives.

### MOF-Encapsulated Vaccines

The vaccine compositions of the disclosure are encapsulated by a MOF protective shell comprising metal ions or clusters coordinated to organic ligands.

Suitable metal ions can be selected from Group 1 through 16 metals of the IUPAC Periodic Table of the Elements including actinides, and lanthanides, and combinations thereof. The metal ion may be selected from Na⁺, K⁺, Mg²⁺, Ca²⁺, Mo⁶⁺, Mo³⁺, Fe³⁺, Fe²⁺, Cu²⁺, Cu⁺, Zn²⁺, Al³⁺, and combinations thereof.

Suitable metal ion coordinating organic ligands can be derived from oxalic acid, malonic acid, succinic acid, glutaric acid, phtalic acid, isophtalic acid, terephthalic acid, citric acid, trimesic acid, 1,2,3-triazole, pyrrodiazole, imidazole or squaric acid.

Metal ions and organic ligands used to construct MOF encapsulated vaccines with good biocompatibility are preferred, for example, sodium, potassium, calcium, iron, zinc, copper, zirconium, titanium, magnesium, manganese, molybdenum, molybdenum or aluminium.

In some embodiments, MOFs are selected from mixed component MOFs, known as MC-MOFs. MC-MOFs have a structure that is characterised by more than one kind of organic ligand and/or metal. MC-MOFs can be obtained by using different organic ligands and/or metals directly in the solution into which MOF precursors and biomolecule are combined, or by post-synthesis substitution of organic ligands and/or metals species of formed MOFs.

In some embodiments, the MOF is a zinc imidazolate framework (ZIF). ZIFs are a sub-class of MOFs that particularly suited to biologic applications. ZIF frameworks feature tetrahedrally-coordinated transition metal ions (e.g., Fe, Co, Cu, Zn) connected by organic imidazolate organic ligands, resulting in three-dimensional porous solids.

Accordingly, MOFs that may be made in accordance with the invention may be carboxylate-based MOFs, heterocyclic azolate-based MOFs, metal-cyanide MOFs. Specific examples of MOFs that may be made according to the present invention include those commonly known in the art as CD-MOF-1, CD-MOF-2, CD-MOF-3, CPM-13, FJI-1, FMOF-1, HKUST-1, IRMOF-1, IRMOF-2, IRMOF-3, IRMOF-6, IRMOF-8, IRMOF-9, IRMOF-13, IRMOF-20, MIL-101, MIL-125, MIL-53, MIL-88 (including MIL-88A, MIL-88B, MIL-88C, MIL-99D series), MOF-5, MOF-74, MOF-177, MOF-210, MOF-200, MOF-205, MOF-505, MOROF-2, MOROF-1, NOTT-100, NOTT-101, NOTT-102, NOTT-103, NOTT-105, NOTT-106, NOTT-107, NOTT-109, NOTT-110, NOTT-111, NOTT-112, MOTT-113, NOTT-114, NOTT-140, NU-1000, rho-ZMOF, PCN-6, PCN-6′, PCN9, PCN10, PCN12, PCN12′, PCNI4, PCN16, PON-17, PCN-21, PCN46, PCN66, PCN68, PMOF-2(Cu), PMOF-3, SNU-5, SNU-15, SNU-215, SNU-21H, SNU-50, SNU-77H, UiO-66, UiO-67, TUDMOF-1, UMCM-2, UMCM-150, UTSA-20, ZIF-2, ZIF-3, ZIF-4, ZIF-8, ZIF-9, ZIF-10, ZIF-11, ZIF-12, ZIF-14, ZIF-20, ZIF-21, ZIF-23, ZIF-60, ZIF-61, ZIF-62, Z1F-64, Z1F-65, ZIF-67, ZIF-68, ZIF-69, ZIF-70, ZIF-71, ZIF-72, ZIF-73, ZIF-74, ZIF-75, ZIF-76, ZIF-77, or ZIF-90.

The MOF-encapsulated vaccine compositions provided herein advantageously may be characterized as having improved stability. As used herein, “improved stability” of a vaccine composition may be determined by using a tissue culture infective assay (TCID50) after storage for a given time and temperature.

For example, the vaccine composition may be characterized by an antigen having improved stability in the composition over one month as compared to a comparative composition comprising the antigen without MOF encapsulation, over three months as compared to such a comparative composition, over six months as compared to such a comparative composition, over nine months as compared to such a comparative composition, or over one year as compared to such a comparative composition.

In some embodiments, the stability of the composition is shown by the relative activity of the antigen after storage at room temperature or at elevated temperatures of up to 40° C. as compared to the initial activity of the antigen. For example, the stability of the composition may be characterized by the antigen maintaining at least 10% of its activity after three months of storage at temperatures up to 40° C., at least 20% of its activity after three months of storage at temperatures up to 40° C., at least 30% of its activity after three months of storage at temperatures up to 40° C., at least 40% of its activity after three months of storage at temperatures up to 40° C., at least 50% of its activity after three months of storage at temperatures up to 40° C., at least 60% of its activity after three months of storage at temperatures up to 40° C., at least 70% of its activity after three months of storage at temperatures up to 40° C., at least 75% of its activity after three months of storage at temperatures up to 40° C., at least 80% of its activity after three months of storage at temperatures up to 40° C., or at least 90% of its activity after three months of storage at temperatures up to 40° C.

In some embodiments, the stability of the composition may be characterized by the antigen maintaining at least at least 10% of its activity after three months of storage at 37° C., at least 20% of its activity after three months of storage at 37° C., at least 30% of its activity after three months of storage at 37° C., at least 40% of its activity after three months of storage at 37° C., at least 50% of its activity after three months of storage at 37° C., at least 60% of its activity after three months of storage at 37° C., at least 70% of its activity after three months of storage at 37° C., at least 75% of its activity after three months of storage at 37° C., at least 80% of its activity after three months of storage at 37° C., or at least 90% of its activity after three months of storage at 37° C.

### Methods of Manufacture

The vaccine formulations described herein are generally prepared by biomimetic mineralization of the vaccine to encapsulate it within a MOF shell.

The method of the invention comprises combining in a solution the vaccine or vector and MOF precursors.

MOF precursors include those compounds known in the art that provide the metal ions described herein in the solution within a suitable solvent. Those compounds may be salts of the relevant metal ions, including metal hydroxides, chlorides, oxychlorides, -nitrates, oxynitrates, -acetates, acetoacetates, -sulphates, trisulphates, hydrogen sulphates, -bromides, -carbonates, -phosphates, and derivatives thereof, including mono- and poly-hydrate derivatives.

Examples of suitable metal salt precursors include, but are not limited to, cobalt nitrate (Co(NO₃)₂·xH₂O), zinc nitrate (Zn(NO₃)₂·xH₂O), iron(III) nitrate (Fe(NO₃)₃·xH₂O), aluminium nitrate (AI(NO₃)₃·xH₂O), magnesium nitrate (Mg(NO₃)₂·xH₂O), calcium nitrate (Ca(NO₃)₂·xH₂O), europium nitrate (Eu(NO₃)₃·xH₂O), dysprosium nitrate (Zn(NO₃)₂·xH₂O), erbium nitrate (Er(NO₃)₂·xH₂O), gallium nitrate (Ga(NO₃)₃·xH₂O), gadolinium nitrate (Gd(NO₃)₃·xH₂O), manganese(II) nitrate (Mn(NO₃)₂·xH₂O), zinc chloride (ZnCl₂·xH₂O), iron(III) chloride (FeCl₂·xH₂O), iron(II) chloride (FeCl₂·xH₂O), aluminium chloride (AlCl₃·xH₂O), magnesium chloride (MgCl₂·xH₂O), calcium chloride (CaCl₂·xH₂O), gallium chloride (GaCl₃·xH₂O), gadolinium chloride (GdCl₃·xH₂O), manganese(II) chloride (MnCl₂·xH₂O), cobalt acetate (Co(CH₃COO)₂·xH₂O), zinc acetate (Zn(CH₃COO)₂·xH₂O), iron(III) acetate (Fe(CH₃COO)₃·xH₂O), iron(O) acetate (Fe(CH₃COO)₂·xH₂O), aluminium acetate (Al(CH₃COO)₃·xH₂O), magnesium acetate (Mg(CH₃COO)₂·xH₂O), calcium acetate (Ca(CH₃COO)₂·xH₂O), gallium acetate (Ga(CH₃COO)₃·xH₂O), gadolinium acetate (Gd(CH₃COO)₃·xH₂O), cobalt sulphate (CoSO₄·xH₂O), zinc sulphate (ZnSO₄·xH₂O), iron(III) sulphate (Fez(SO₄)₃·xH₂O), iron(II) sulphate (FeSO₄·xH₂O), aluminium sulphate (Al₂(SO₄)₃·xH₂O), magnesium sulphate (MgSO₄·xH₂O), calcium sulphate (CaSO₄·xH₂O), nickel sulphate (NiSO₄·xH₂O), lead sulphate (PhSO₄·xH₂O), cadmium sulphate (CdSO₄·xH₂O), manganese(II) sulphate (MnSO₄·xH₂O). cobalt hydroxide (Co(OH)₂·xH₂O), zinc hydroxide (Zn(OH)₂·xH₂O), iron(III) hydroxide (Fe(OH)₃·xH₂O), iron(III) oxide:hydroxide (FeO(OH) xH₂O), iron(II) hydroxide (Fe(OH)₂·xH₂O), aluminium hydroxide (Al(OH)₃·xH₂O), magnesium hydroxide (Mg(OH)₂·xH₂O), calcium hydroxide (Ca(OH)₂·xH₂O), manganese(II) hydroxide (Mn(OH)₂·xH₂O), cobalt bromide (CoBr₂·xH₂O), zinc bromide (ZnBr₂·xH₂O), iron(III) bromide (FeBr₃·xH₂O), iron(II) bromide (FeBr₂·xH₂O), aluminium bromide (AlBr₃·xH₂O), magnesium bromide (MgBr₂·xH₂O), calcium bromide (CaBr₂·H₂O), gallium bromide (GaBr₃·xH₂O), gadolinium bromide (GdBr₃·xH₂O), manganese(II) bromide (MnBr₂·xH₂O), cobalt carbonate (CoCo₃·xH₂O), zinc carbonate (ZnCo₃·xH₂O), iron(III) carbonate (Fez(CO₃)₃·xH₂O), aluminium carbonate (Al₂(CO₃)₃·xH₂O), magnesium carbonate (MgCO₃·xH₂O), calcium carbonate (CaCO₃·xH₂O), gallium carbonate (Ga₂(CO₃)₃·xH₂O), gadolinium carbonate (Gd₂(CO₃)₃·xH₂O), manganese(II) carbonate (MnCO₃·xH₂O), and mixtures thereof, where x ranges range from 0 to 12.

MOF precursors also include organic ligands of the kind described herein that coordinate the metal ion clusters in the MOF framework. The organic ligands include molecules that have at least two chemical moieties capable of coordinating a metal ion. In some embodiments, these groups comprise carboxylates, phosphonates, sulphonates, N-heterocyclic groups, and combinations thereof.

Examples of organic ligand precursors include, but are not limited to, α-cyclodextrin, β-cyclodextrin, γ-cyclodextrin, oxalic acid, oxalate, fumaric acid, fumarate, maleic acid, maleate, 4,4,4″-[benzene-1,3,5-triyl-tris(ethyne-2,1-diyl)]tribenzoate, biphenyl-4,4′-dicarboxylate, 4,4,4″-[benzene-1,3,5-triyl-tris(benzene-4,1-diyl)]tribenzoate, 1,3,5-benzenetribenzoate, 1,4-benzenedicarboxylate, benzene-1,3,5-tris(1H-tetrazole), 1,3,5-benzenedicarboxylic acid, terephthalic acid, imidazole, benzimidazole, 2-nitroimidazole, 2-methylimidazole (Hmlm), 2-ethylimidazole, 5-chloro benzimidazole, 1,4-Bis(1-imidazolyl)benzene), 4,4,-Bispyridyl, 1,4-purine, fumaric acid, Diazabicyclo[2,2,2]octane, 2-amino-1,4-benzenedicarboxylic, 2-amino-1,4-benzenedicarboxylic acid, 4,4′-Azobenzenedicarboxylate, 4,4′-Azobenzenedicarboxylic acid, Aniline-2,4,6-tribenzoate, Amiline-2,4,6-tribenzic acid, Biphenyl-4,4′-dicarboxylic acid, 1,1′-Biphenyl-2,2′,6,6′-tetracarboxylate, 1,1′-Biphenyl-2,2′,6,6′-tetracarboxylic acid, 2,2′-Bipyridyl-5,5′-dicarboxylate, 2,2-Bipyridyl-5,5′-dicarboxylic acid, 1,3,5-Tris(4-carboxyphenyl)benzene, 1,3,5-Tris(4-carboxylatephenyl)benzene, 1,3,5-Benzenetricarboxylate, 2,5-Dihydroxy-1,4-benzenedicarboxylate, 2,5-Dihydroxy-1,4-benzenedicarboxylic acid, 2,5-Dimethoxy-1,4-benzenedicarboxylate, 2,5-Dimethoxy-1,4-benzenedicarboxylic acid, 1,4-Naphthalenedicarboxylate, 1,4-Naphthalenedicarboxylic acid, 1,3-Naphthalenedicarboxylate, 1,3-Naphthalenedicarboxyluc, acid, 1,7-Naphthalenedicarboxylate, 1,7-Naphthalenedicarboxylic acid, 2,6-Naphthalenedicarboxylate, 2,6-Naphthalenedicarboxylic acid, 1,5-Naphthalenedicarboxylate, 1,5-Naphthalenedicarboxylic acid, 2,7-Naphthalenedicarboxylate, 2,7-Naphthalenedicarboxylic acid, 4,4′,4′-Nitrilotrisbenzoate, 4,4′,4″-Nitrilotrisbenzoic acid, 2,4,6-Tris(2,5-dicarboxylphenylamino)-1,3,5-triazine, 2,4,6-Tris(2,5-dicarboxylatephenylamino)-1,3,5-triazine, 1,3,6,8-Tetrakis(4-carboxyphenyl)pyrene, 1,3,6,8-Tetrakis(4-carboxylatephenyl)pyrene, 1,2,4,5-Tetrakis(4-carboxyphenyl)benezene, 1,2,4,5-Tetrakis(4-carboxylatephenyl)benzene, 5,10,15,20-Tetrakis(4-carboxyphenyl)porphyrin, 5, 10, 15,20-Tetrakis(4-carboxylatephenyl)porphyrin, adenine, adeninate, fumarate, 1,2,4,5-benzenetetracarboxylate, 1,4,5-benzenetetracarboxylic acid, 1,3,5-benzenetribenzoic acid, 3,-amino-1,5-benzenedicarboxylic acid, 3-amino-1,5-benzenedicarboxylate, 1,3-benzenedicarboxylic acid, 1,3-benzenedicarboxylate, 4′,4′,4″-[benzene-1,3,5-triyl-tris(ethyne-2,1-diyl)]tribenzoic acid, 4,4′,4″-[benzene-1,3,5-triyl-tris(benzene-4,1-diyl)]tribenzoic acid, trans.trans-muconic acid, trans, trans-muconate, cis, trans-muconic acid, cis,trans-muconate, cis,cis-muconic acid, cis,cis-muconate, pyrazole, 2,5-dimethylpyrazole, 1,2,4-triazole, 3,5-dimethyl-1,2,4-triazole, pyrazine, 2,5-dimethylpyrazine, hexamethylentetraamine, nicotinic acid, nicotinate, isonicotinic acid, isonicotinate, 4-(3,5-dimethyl-1H-pyrazole)-benzoic acid, 2,5-furandicarboxylic acid, 2,5-furandicarboxylate, 3,5-dimethyl-4-carboxypyrazole, 3,5-dimethyl-4-carboxylatepyrazole, 4-(3,5-dimethyl-1H-pyrazol-4-yl)-benzoic acid, 4-(3,5-dimethyl-1H-pyrazol-4-yl)-benzoate, and mixtures thereof.

It will be understood that the organic ligands can also be functionalised organic ligands. For example, any one of the organic ligands described herein may be additionally functionalised by amino-, such as 2-aminoterephthalic acid, urethane acetamide-, or amide-. The organic ligand can be functionalised before being used as precursor for MOF formation, or alternatively the assembled MOF itself can be chemically treated to functionalise its bridging organic ligands.

A skilled person will be aware of suitable chemical protocols that allow functionalizing MOFs with functional groups, either by pre-functionalizing organic ligands used to synthesize MOFs or by post-functionalizing pre-formed MOFs.

Suitable functional groups that may be provided on the MOF include —NHR, —N(R)₂, —NH₂, —NO₂, —NH(aryl), halides, aryl aralkyl, alkehyl, alkynyl, pyridyl, bipyridyl, terpyridyl, anilino, —O(alkyl), cycloalkyl, cycloalkenyl, cycloalkynyl, sulfonamide, hydroxyl, cyano, —(CO)R, —(SO₂)R, —(CO₂)R, —SH, —S(alkyl), —SO₃H, —SO₃—, M₊, —COOH, COO-M+, —PO₃H₂, —PO₃H-M⁺, —PO₃²—M²⁺, —CO₂H, silyl derivatives, borane derivatives, ferrocenes and other metallocenes, where M is a metal atom, and R is C₁₋₁₀ alkyl.

There are no particular restrictions on the solvents that can be used to prepare the solution in which MOF precursors and vaccine are combined, provided that (i) the MOF precursors are soluble in the solvent, and (ii) the vaccine is compatible with the solvent. That is, the solvent will typically be one that does not adversely affect the bioactivity of the one or more antigens. Preferably the solvent, is biocompatible.

Examples of solvent that may be used include methanol, ethanol, dimethyl sulfoxide (DMSO), acetone, water and mixtures thereof. Preferably, the solvent is water.

In some embodiments, the solution into which the one or more antigens and MOF precursors are combined is an aqueous solution, for example deionised water, or a physiological buffered solution (water comprising one or more salts such as KH₂PO₄, NaHaPO⁺, K₂HPO₄, Na₂HPO₄, Na₃PO₄, K₃PO₄, NaCl, KCl, MgCl₂, CaCl₂), etc.).

Provided the MOF forms, there is no particular limitation regarding the concentration of MOF precursors present in the solution. The skilled person will appreciate that the minimum precursor concentration is determined by the self-assembly of MOF and is dependent on the MOF type. Also, the maximum precursor concentration is determined by the toxicity of the MOF precursors to the vaccine or vector being encapsulated. And its biocompatibility and environmental toxicity. This further depends on, for example, the type of vaccine or vector, the mode and formulation type (released from the MOF or intact) of administration, and/or the minimum vaccine titre required to be administered, etc. For example, the minimum exemplified working precursor concentration for a virus titre of 10⁹ in 200 μl volume was 80 mM 2-methylimidazole (Hmlm) and 20 mM ZnAc (ZIF-8). The maximum precursor concentration exemplified was 640:160, limited by the toxicity of MOF in in vitro assays to the DF1 cells used. For aluminium fumarate, the maximum concentrations for the precursors was 50 mM, limited by the solubility of fumaric acid, the minimum was as low as 5 mM.

Concentrations of MOF precursors in the solution can include a range between about 0.001 M and 1 M, between about 0.01 M and 0.5 M, between about 0.01 and 0.2 M, between about 0.02 M and 0.2 M, between about 0.02 M and 0.15 M, between about 0.05 M and 0.15 M between about 0.08 M and 0.16 M. The values refer to concentration of organic ligand as well as concentration of metal salt, relative to the total volume of the solution containing the MOF precursors and the one or more antigens.

The ratio between the concentration of organic ligands and the concentration of metal salts is not limited, provided the ratio is adequate for the formation of MOF promoted by the combination with the one or more antigens in accordance to the invention. In some embodiments, the organic ligand to metal salt ratio may range from 100:1 to 1:100, from 60:1 to 1:60 (mol:mol), from 30:1 to 1:30, from 10:1 to 1:10, torn 5:1 to 1:5, from 2.5:1 to 1:2.5, from 2:1 to 1:2, or from 1.5:1 to 1:1.5.

Suitable concentrations of protein in the solution can include a range of between about 0.1 and 20 mg/mL, between about 0.15 and 10 mg/mL between about 0.15 and 7.5 mg/mL, between about 0.2 and 5 mg/mL, between about 0.25 and 5 mg/mL, between about 0.03 and 5 mg/mL, between about 0.025 and 2.5 mg/mL, between about 0.025 and 2 mg ml, between about 0.025 and 1.5 mg/mL, or between about 0.025 and 1.25 mg/mL.

According to the method of the invention, the vaccine or vector promotes formation of the encapsulating MOF framework.

By the vaccine or vector ‘promotes’ formation of the encapsulating framework is meant the vaccine or vector per se causes, induces or triggers formation of the MOF framework upon combination with the MOF precursors in a solution. As a result of the one or more antigens promoting formation of the framework, the MOF framework forms around the vaccine or vector antigens to eventually encapsulate it within a MOF outer shell.

Without being limited to theory, it is believed the vaccine or vector induced formation of MOF may be related to the charge, hydrophilicity/hydrophobicity nature or chelating ability of the specific vaccine or vector. It is believed that formation of encapsulating MOF is facilitated by the vaccine or vector affinity towards MOF precursors arising, for example, from intermolecular hydrogen bonding and hydrophobic interactions.

The resulting increase in the local concentration (i.e., in the immediate surroundings of the vaccine or vector) of both metal cations (deriving from the dissolution of the metal salt precursor) and organic ligands would facilitate pre-nucleation clusters of MOF framework.

It has been found that hydrophilic molecules and molecules having negatively charged domains or moieties (e.g. carboxyl groups, hydroxyl groups, amino groups etc) show improved ability to nucleate MOFs over molecules with more hydrophobic character and positively charged moieties. It may therefore be hypothesised that negatively charged domains in the vaccine or vector attract the positive metal ions provided by the MOF metal precursor in solution and contribute to stabilize the metal-organic ligand clusters at the early stages of MOF formation.

In some embodiments, combining the MOF precursors in solution with the vaccine or vector is surprisingly sufficient to cause formation of the MOF framework. There is no need to apply other factors or reagents to trigger formation of the MOF. For example, it is not necessary to apply heat to the solution as conventionally done in traditional solvothermal MOF synthesis methods (which typically require use of a heat source such as an oven, for example a microwave oven, a hot plate, or a heating mantel). For example, for ZIF-8, which is composed of 2-methylimidazole (Hmlm) and Zn2+, the MOF shell can self-assemble spontaneously in water around the vaccine or vector (at low precursor concentrations) without the application of external energy such as heat, pressure or even additional time. At higher precursor concentrations, the precursors can precipitate with or without the presence of the vaccine or vector.

Accordingly, in some embodiments formation of the encapsulating framework is effected at a solution temperature that is lower than 75° C., 50° ° C., or 35° ° C. Thus, the solution temperature may be between 2° C. and 75° C., between 2° C. and 60° C., between 16° C. and 27° C., or between 18° C. and 25° C. The skilled person will appreciate that the temperature used is dependent on the MOF type and the heat sensitivity of the vaccine or vector.

In some embodiments, the method is performed at ambient room temperature. For example, for aluminium fumarate, which is composed of fumaric acid and sodium aluminate, the MOF shell can self-assemble around the vaccine or vector at ambient room temperature. Performing the method at ambient room temperature or below is advantageous for heat sensitive vaccines or vectors.

There is no particular limitation on the order in which the MOF precursors and the vaccine or vector may be combined into the solution.

For example, a solution containing a metal precursor may be first mixed with a solution comprising an organic ligand, and a separate solution comprising a vaccine or vector is subsequently introduced into the solution containing the metal salt and the organic ligand. Alternatively, a solution comprising a vaccine or vector and an organic ligand may be first prepared, and subsequently introduced into a separate solution comprising a metal precursor.

Also, a solution comprising a vaccine or vector and a metal precursor may be first prepared, and subsequently introduced into a separate solution comprising an organic ligand.

Still further, separate solutions each individually comprising a metal precursor, an organic ligand and a vaccine or vector, respectively, may be mixed together at the same time.

In one embodiment, the vaccine or vector is introduced into a solution comprising the MOF precursors.

Formation of MOF shell according to the method of the invention is advantageously fast. Depending on the vaccine or vector used and the type of MOF precursors used, it has been found that upon bringing the vaccine or vector and the MOF precursors together in a solution MOF may form within about 1 second, 10 seconds, 1 minute, 10 minutes, 30 minutes, 60 minutes or 2 hours. Under the same conditions of time, temperature and concentration of MOF precursors, it was found in a solution containing only MOF precursors (i.e. with no vaccine or vector) MOF would not form. In other words, the vaccine or vector per se has been found to promote formation of MOF.

The resultant antigen encapsulated MOF solution may be dried at any suitable temperature and pressure conditions, which preferably are selected to maintain the physical stability and/or chemical stability and/or biological activity of the vaccine or vector. In a preferred embodiment, the aqueous solution is dried at an ambient temperature for a time sufficient to form the dry solid form of the vaccine composition. For example, the aqueous solution may be dried at ambient temperature for a period from about 30 minutes to about one week to form the dry solid vaccine formulation, for example, from about 45 minutes to about one week, or from about one hour to about one week, or from about one hour to about one day. In other embodiments, the aqueous solution may be vacuum-dried or dried using a combination of air-drying and vacuum-drying. Although various temperatures and humidity levels can be employed to dry the aqueous solution, the formulations preferably are dried at temperature from −80° ° C. to 60° C. (e.g., from 15° C. to about 45° C., from about 25° C. to about 45° C., or at about ambient temperature) and 0 to 10% relative humidity. In some embodiments, one or more excipients are added to the solution prior to drying, for example, skim milk. Such excipients may protect the MOF shell during drying, for example, by inducing a variation of the pH of the solvent.

Examples of MOFs that may be used in applications based on pH-triggered release of the one or more antigens include MOFs that are stable at certain pH values, but dissolve at certain other pH values. For example, the MOF may be stable above a threshold pH value. In that case there is no detectable release of the vaccine or vector into the solution within which the MOF is suspended. However, the MOF may dissolve when the pH drops below the threshold, resulting in the release of the vaccine or vector into the solution. For example, certain ZIFs are stable at extracellular pH (about 7.4), but dissolve when the pH drops below 6.5, for example, at intracellular pH (about 6).

In this context, the stability of a MOF in a solvent at a certain pH is determined in relation to the amount of metal ions released into the solvent by the MOF when dissolving. The concentration of metal ions in the solvent is determined by Inductively Coupled Plasma (ICP) performed before and after exposure of the MOF to that pH condition for 2 hours. A MOF will be deemed ‘stable’ if the measured concentration of metal ion in solution after 2 hours differs by less than of 15% from the initial value.

### Methods of Administration

The vaccine formulations provided herein may be administered to a subject or patient by any suitable means. As used herein, the term “patient” typically refers to a child or adult human in need of vaccination.

The vaccine composition may be reconstituted in a physiologically acceptable liquid vehicle to form an injectable solution or suspension, and then the injectable solution or suspension is injected into the subject or patient. The vaccine formulation may be reconstituted directly in a hypodermic syringe or in a sterile vial or other container. The reconstituted vaccine composition then may be injected into the subject or patient, for example, by intramuscular, intradermal, or subcutaneous injection.

In some embodiments, the vaccine or vector is released from the MOF shell by pH-induced targeted release prior to administration to the subject or patient. For example, the vaccine composition may be reconstituted in a physiologically acceptable liquid vehicle having a pH at which the MOF shell dissolves.

### EXAMPLES

### Materials

All materials, zinc acetate dihydrate (>99%), 2-methylimidazole (99%), sodium aluminate (>99%), fumaric acid (>98%), bovine serum albumin (BSA, 98%), phosphate buffered saline (PBS) tablets, sodium citrate dihydrate, citric acid, EDTA disodium salt (>99%), hydrochloric acid, methanol and ethanol were purchased commercially and used as is. Ultrapure water (MilliQ, 18.2 MQ at 25° C.) was used to make aqueous solutions. All buffers and water were sterilized by autoclaving at 121° C. for 1 h.

### Cell Culture

DF1 (chicken fibroblast) cells were maintained at 37° ° C., 5% CO₂ in complete cell culture medium containing DMEM (Glutamax), 10% fetal calf serum (FCS) and 1% Pen/Strep (100 μg/mL penicillin and 100 units/mL streptomycin) and subcultured approximately every 4 d. One day prior to the TCID50 infection, cells were seed at a density of 2.5×10⁴ cells per well in 96 well plates using seeding medium containing DMEM (Glutamax) with 1% FBS and 1% Pen/strep. For immunofluorescence staining, a CSIRO manufactured monoclonal antibody (mAb) against the hemagglutinin-neuraminadase (HN) protein of NDV was used as the primary antibody at a 1:20 dilution followed by Alexa Fluor 488 goat anti-mouse IgG (H+L) secondary antibody at a 1:400 dilution.

### Preparation of Standard ZIF-8

Separate solutions of 2-methylimidazole (160 mM, 525.2 mg) and zinc acetate dihydrate (40 mM, 351.2 mg) were each prepared in 40 mL of methanol at room temperature, respectively. The two solutions were mixed by agitation for about 20 s and let to sit at room temperature ageing over a period of 24 h to grow ZIF-8 crystals. ZIF-8 crystals were then collected using centrifugation at 5000 g for 10 min and washed 3 times with methanol.

### Preparation of MOF@Vaccine Composites

Metal Organic framework (MOF) encapsulation of live viral vaccines NDV V4 strain and Influenza virus A/WSN/H1N1 strain used for this work were each prepared by propagation in serum-pathogen free fertilised chicken eggs. The allantoic fluid containing the viruses were harvested and aliquoted before transfer to −80° C. for long term storage.

ZIF-8 encapsulation of Newcastle disease virus V4 (NDV); ZIF-8@NDV NDV was removed from −80° C. storage and let to thaw at 4° C. for approximately 4 h. To this freshly thawed NDV containing fluid, 100 μL of 2 methylimidazole (Hmlm) solution (320 mM) was added and carefully mixed by pipetting. Next, a 100 μL solution of Zinc acetate (ZnAc) dihydrate (80 mM) was quickly added and mixed using soft pipetting. Flocculates appeared immediately and the solution was left to sit in the BSC II over a period of 30 min at room temperature. The pellet was collected by centrifugation at 7000g for 10 min, the supernatant was collected for virus titre assessment and the pellet was washed with water followed by collection of ZIF-8@NDV using centrifuging as before. A higher precursor concentration, of 640 mM HmIm and 160 mM Zinc acetate were also assessed. The pellet was used as it is for encapsulation assessment and lyophilised as detailed below for storage and stability testing. Throughout the study, the working volumes were maintained for comparison between the control and test samples, i.e. when a 200 μL of starting virus solution was used for the MOF@vaccine synthesis, the resulting pellet was reconstituted to make a 200 μL volume before infecting cells to perform the TCID50 assay.

### ZIF-8 Encapsulation of Influenza a Virus H1N1 (WSN); ZIF-8@WSN

Using the Influenza A virus strain WSN (A/WSN/1933(H1N1), ZIF-8@WSN was prepared by applying the same methodology and precursor concentrations as detailed for ZIF-8@NDV. Application of a higher precursor concentration of 640 mM Hmlm and 160 mM Zinc acetate demonstrated higher encapsulation efficiency for WSN. The pellet was used as is for encapsulation assessment.

### Aluminium Fumarate (AlFum) Encapsulation of WSN; AlFum@WSN

Three methods, ‘M1’, ‘M2’ and ‘M3’ were tested for AlFum@WSN synthesis. M1: To 300 UL of WSN in allantoic fluid, 300 UL of sodium aluminate solution (45 mM) was added and carefully mixed by pipetting. To this, 300 μL of fumaric acid solution (45 mM) was quickly added and carefully mixed by pipetting. Immediate flocculation was observed. In the alternative M2 synthesis, the order of addition of the precursor solutions were reversed. The addition of fumaric acid to WSN followed by sodium aluminate was assessed. In the method M3, 600 L of sodium aluminate solution (45 mM) was added to 300 UL of WSN in allantoic fluid followed by 600 UL of fumaric acid solution (45 mM). In all methods, the reaction solution was left to sit in the BSC II over a period of 30 min at room temperature. The pellet was collected by centrifugation at 7000g for 10 min, the supernatant was collected for virus titre assessment and the pellet was washed with water followed by collection of Alfum@WSN using centrifuging as before. The pellet was used as it is for encapsulation assessment.

### Freeze Drying of ZIF-8@NDV

The hydrated ZIF-8@NDV pellet was lyophilised for storage and stability testing. Briefly, the pellet obtained post-centrifugation was placed at −80° C. for 15 min to let it freeze. It was then and transferred to the Labconco Triad Lyophilizer with the collector set to −82° C. The samples were dried over a period of about 20 h using a three-step program. The pre-freeze step was set at −72° C. for 7 h, followed by a drying segment 1 set at −45° C. for 9 h and segment 2 set at +23° C. for 1.5 h. Obtained powders were immediately septum cap sealed under vacuum. The addition of excipients Trehalose (T) and Skim milk (SM) and their combinations thereof in ZIF-8@NDV synthesis to protect the composite from FD stress was assessed.

### Vaccine Potency Assay In Vitro

Recovery from the MOF

Recovery herein means the exfoliation of the MOF@Vaccine composites using a release buffer that degrades the MOF while preserving the virus infective titre. For ZIF-8@NDV and ZIF-8@WSN composites, a 50 mM Sodium citrate buffer (pH 5.0) was added to the pellets, gently mixed by pipetting and left for about 2-5 minutes at room temperature till the wet-samples appeared clear. Released samples were used immediately for TCID50 assay after release to assess infectious titre. Dry ZIF-8@NDV when resuspended in the release buffer required longer time (10-15 min) for dissolution. WSN from Alfum@WSN was released using 100 mM EDTA solution (pH 7.0). The release buffers were added to make a final volume of either 200 μL or 300 μL, respectively to match the initial volume (and titre) of virus used for MOF@Vaccine synthesis.

### Fluorescent TCID50 Assay

A tissue culture infectious dose (TCID50) assay was conducted to determine the degree of vaccine potency retention following the various MOF@Vaccine composite preparations, FD methods and storage conditions. TCID50 is the dilution at which 50% of the test cell monolayers show evidence of infectivity by the virus. The wet pellet or dry lyophilised virus and MOF@vaccine composites were recovered and reconstituted using the required release buffer solution. Control virus was removed immediately prior to testing from the −80° C. storage and left to thaw at 4° C. The test samples were serially diluted in 1% FCS cell culture medium using a 96 well plate, starting from 10-fold dilution to the lowest 10¹¹-fold dilution of the sample, in quadruplicate. The infection procedure required addition of 50 μL dilutions to a 96 well plate containing 2.5×10⁴ DF1 cells per well. The DF1 cells were seeded in 100 μL of FCS deficient (1%) cell culture media and left to adhere for 24 h before infection. After 5 d of incubation in a cell culture incubator at 37° C., 5% CO₂, the plate was examined under a microscope to enumerate the number of wells exhibiting cytopathic effect (CPE) for WSN samples. For NDV, the plate was stained for presence of viral protein using a monoclonal antibody followed by a fluorescently labelled secondary antibody to detect the presence of replicating virus. TCID50 was calculated using the Reed and Muench method (Am. J. Epidemiol. 1938, 27, 493-497).

### Immunofluorescence Staining

NDV and ZIF-8@NDV infected cell culture plates were immunofluorescence stained to visualize the number of infected wells. The infected plate post-incubation for 5 d at 37° C., 5% CO₂ was moved into the BSC II and the culture media was carefully removed. The plate was rinsed with phosphate buffered saline (PBS) preheated to 37° C. It was then fixed with 4% paraformaldehyde (PFA) solution (300 μl each well) for 1 h at 37° C. PFA was removed, and the plate was blocked for unspecific antibody binding using 2% FCS in PBS (2% PBSA) for 30 min at room temperature followed by permeabilization using 0.01% Triton X-100 solution. The cells were washed once using 2% PBSA before addition of the primary antibody (1:20) diluted in 2% PBSA and incubation at 4° C. for 1 h. The cells were then washed 3 times for 5 min each using 2% PBSA before addition of the secondary antibody (1:400) diluted in 2% PBSA and incubation in dark at 4° C. for 30 min. The plate was then washed twice with 2% PBSA and twice with tissue culture water before incubation with DAPI stain (1:2000) diluted in PBS for 10 min at room temperature. The plate was then washed twice with tissue culture water before reading at the microscope or storage in dark at 4° C. Cells were kept hydrated under a small volume of PBS and plates were read within 7 d of staining.

### Storage Stability Studies

Aliquoted, lyophilised preparations of NDV; NDV+T/SM and ZIF-8@NDV+T/SM, and freshly thawed aliquots of NDV in allantoic fluid were placed at 4° C., room temperature (regulated at approximately 25° C.) and 37° C. for a period of 12 weeks. At regular time intervals, virus infectivity was measured by TCID50 assay on three samples (n=3) from each formulation type stored at each of the three temperature conditions, respectively.

### Characterisation Techniques

### Fluorescence Imaging

Fluorescence images were captured using the EVOS FL Imaging system with high resolution CMOS camera. The Alexa-fluor488 labelled secondary antibody facilitated fluorescent green labelling for viral antigen and cell nuclei were labelled blue using DAPI stain.

### Scanning Electron Microscopy (SEM)

Samples were mounted either on carbon tape or silicon wafer and then put on aluminium stubs. The samples were coated with conductive iridium using Cressington HR sputter coater for 20 seconds to give a 3 nm coating. Samples were imaged using a Zeiss Merlin FESEM at an accelerating voltage of 3 kV in the secondary electron or in lens modes depending on magnification. Magnification is indicated by the scale bars in each image.

### Elemental Composition

Elemental dispersive spectroscopy (EDS) was conducted using Oxford Instruments Extreme windowless SSD detector at an accelerating voltage of 5 kV.

### Powder X-Ray Diffraction

Dry samples were briefly ground in an agate mortar and pestle prior to being loaded onto zero background plate sample-holders for data collection. A Bruker D8 Advance X-ray Diffractometer operating under CuKα radiation (40 kV, 40 mA) equipped with a LynxEye detector was employed to obtain the XRD pattern. The sample was scanned over the 20 range 5-85° with a step size of 0.02° and a count time of 3.2 seconds per step. 178/192 of the sensor strips on the LynxEye detector were used, to give an equivalent count time of 569.6 seconds per step.

### SAXS Analysis

Data were collected at the Small Angle X-ray Scattering beamline of the Australian Synchrotron. Raw data were averaged from five repeat scans between 0.01-0.97 Å-1 using a wide-range SAXS detector (Pilatus 1M 12 keV, 700 mm camera length). Background scattering was subtracted from sample data. Scatterbrain software was used for both the averaging and the background subtraction process.

### Dynamic Light Scattering (DLS)

Dynamic-light scattering and zeta potential/surface charge were measured with a Malvern Zetasizer Nano ZS. A disposable cuvette was used at 25° C. with a 633 nm laser source, a medium refractive index of 1.33, a material refractive index of 1.51, and a scattering angle of 175°.

### Transmission Electron Microscopy (TEM)

Carbon-coated grids (EMSCF200H-CU-TH, ProSciTech) were glow discharged to render them hydrophilic. A 10 ul drop of sample was applied to an upturned grid held in anti-capillary forceps, over moist filter paper, and left for 10 minutes to adsorb. The excess sample was then removed with filter paper. If stained, the grid was then inverted onto a drop of 2% PTA stain, pH 6.9 on Parafilm, for 1 minute. The grid was removed, the stain wicked away with filter paper and allowed to dry before viewing in the microscope. The samples were examined using a Tecnai 12 Transmission Electron Microscope (FEI, Eindhoven, The Netherlands) at an operating voltage of 120 KV. Images were recorded using a FEI Eagle 4k×4k CCD camera and AnalySIS v3.2 camera control software (Olympus.).

### Thermogravimetric Analysis (TGA)

TGA was conducted using a Mettler TGA/DSC 1 thermal analysis instrument. Data were collected in the temperature range of 25 to 900° C. under a nitrogen flow rate of 0.5 mL min-1. Data were analysed using the TGA STARe evaluation program.

### Statistical Analysis

All values reported are means±SD. As and where indicated, log transformed data were analysed by either unpaired t-tests (*, p<0.05) or one-way ANOVA (p<0.05) followed by Dunett's multiple comparison analysis (*, p<0.05; **, p<0.01, ***, p<0.001, ****, p<0.0001).

### General Metal Organic Framework (MOF) Encapsulation of Live Viral Vaccines

In a typical experiment, for biomimetic mineralised growth of the MOF around the live virus, a frozen stock vial of the virus is slowly thawed from its −80° C. storage to 4° C. in the refrigerator. Once thawed, the aliquot is used immediately, and any remains are disposed. This is done to avoid titre loss in the control and test samples due to the freeze-thaw cycle. Then, an aqueous solution of the organic linker (or the metal salt solution) is added immediately to the freshly thawed virus aliquot, followed by the addition of the metal salt solution (aqueous) or vice versa. In the presence of the virus, the reaction solution instantaneously begins to flocculate indicating MOF initiation which is left to form over a period of 30 min. The MOF@vaccine pellet is collected after centrifugation.

For this proof-of-concept work, ZIF-8 MOF and NDV strain V4 live viral vaccine were used. ZIF-8 was applied to this research because of its biocompatibility, extraordinary thermal and chemical resistance and, the ease of performing linker modifications to enhance bio-interfacial interactions. The commercially available Newcastle Disease Virus (NDV) strain V4, live viral vaccine was used for this work. NDV is one of the live virus vaccines of most demand for the global poultry industry. Recognized ninety years ago, Newcastle disease (ND) is an avian infection that continues to widely affect the economic livelihoods of farmers and the food supplies worldwide.

The MOF encapsulation efficiency was determined by measuring the infective viral titre in both the supernatant and the pellet following release, using an in vitro assessment of median tissue culture infectious dose (TCID50) assay in DF1 cells. It is known that negatively charged proteins increase the local concentration of positively charged metal ions and thereby the organic ligands, facilitating prenucleation cluster formation of ZIF-8 around the biomacromolecules leading to controlled MOF formation, which in turn enhances their stability.

Throughout the study, the working volumes were maintained for comparison between the control and test samples, i.e. when a 200 μl of starting virus solution was used for the MOF@vaccine synthesis, the resulting pellet was reconstituted to make a 200 μl volume before infecting cells to perform the TCID50 assay.

The metal and organic ligand concentrations influence the intrinsic structure and morphology of the MOF and thereby would contribute to the structural and functional integrity of the MOF@Virus composite. A matrix of precursor concentrations and viral titre were tested for optimal synthesis (FIG. 14a). A minimum precursor concentration of 20 mM zinc acetate (ZnAc) and 80 mM 2-methyl imidazole (Hmlm) were required for the biomimetic mineralization of ˜10¹² TCID50/mL NDV titre to occur. Proteins induce the formation of MOF coatings by concentrating the precursors facilitating MOF crystallization around them (K. Liang, R. Ricco, C. M. Doherty, M. J. Styles, S. Bell, N. Kirby, S. Mudie, D. Haylock, A. J. Hill, C. J. Doonan, Nature Commun. 2015, 6, 7240).

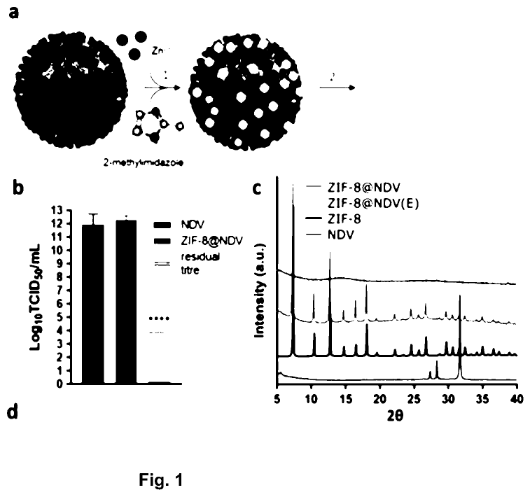

The present inventors' postulate that the same phenomena apply to MOF@Virus formation. Schematic FIG. 1a shows that triggered by the virus' surface proteins the metal ion and organic ligand concentrate around the virions, self-assembling into a MOF coating. Spontaneous flocculation did not occur in absence of the virus confirming the virions initiate the MOF self-assembly at lower precursor concentration. However, at higher precursor concentrations of 160 mM ZnAc and 640 mM Hmlm and beyond, precipitation occurs even in the absence of the virus.

The viral titre in ZIF-8@NDV composites synthesized using ZnAc and Hmlm concentrations of 40:160 and 80:320 mM, respectively was determined using an in vitro fluorescent TCID50 assay (FIG. 14b). The ZIF-8@NDV composite completely retained NDV infectious titre with no reduction in viral titre from the starting titre of 1.8×10¹² TCID50/mL and the presence of significantly small, 1.9×10⁴ TCID50/mL residual titre in the supernatant (FIG. 1b). The structural and functional integrity of NDV following encapsulation into a ZIF-8@NDV system was assessed by releasing the viral load using a sodium citrate buffer (pH 5.0, 50 mM) followed by a quantitative TCID50 assay. The chelating effect of the citrate on the divalent zinc ions, breaks the coordination bonds that hold the ZIF-8 MOF together, releasing the encapsulated virions.

All MOF synthesis and post-processing were performed in water. The aqueous synthesis of ZIF-8 at low molar ratio of Hmlm to Zinc (1:4) favour the formation of an amorphous material (F. Carraro, M. d. J. Velásquez-Hernández, E. Astria, W. Liang, L. Twight, C. Parise, M. Ge, Z. Huang, R. Ricco, X. Zou, Chem. Sci. 2020, 11, 3397-3404; A. F. Ogata, A. M. Rakowski, B. P. Carpenter, D. A. Fishman, J. G. Merham, P. J. Hurst, J. P. Patterson, J. Am. Chem. Soc. 2020, 142, 1433-1442). However, to confirm the composition of the material, a crystalline phase is required. For this, the ZIF-8@NDV composite was washed post-process with a 50% ethanol solution giving a crystalline ZIF-8@NDV(E) composite. FIG. 1c shows similar x-ray diffraction (XRD) patterns for control ZIF-8 material and ZIF-8@NDV(E) confirming the composition, while the aqueous synthesis derived ZIF-8@NDV used in this work is largely amorphous. These results were also confirmed using small angle x-ray spectroscopy (SAXS) analysis on these composites.

The scanning electron microscopy (SEM) images in FIG. 5 shows (i) aggregates of NDV, where individual virions would be greater than 100 nm in size (V. Schirrmacher, Int. J. Mol. Sci. 2017, 18, 1103), (ii) ZIF-8@NDV(E) crystals with typical ZIF-8 rhombic dodecahedral morphology while (iii) ZIF-8@NDV precipitate did not have any particular defined morphology. The MOF phase and its chemical, thermal and mechanical stability contribute to the stability of the MOF@bioactive composite (A. J. Howarth, Y. Liu, P. Li, Z. Li, T. C. Wang, J. T. Hupp, O. K. Farha, Nat. Rev. Mater 2016, 1, 1-15). A recent study demonstrates slower release of biomolecules when they were encapsulated within a crystalline phase of the MOF suggesting its superior stability (F. Carraro, M. d. J. Velásquez-Hernández, E. Astria, W. Liang, L. Twight, C. Parise, M. Ge, Z. Huang, R. Ricco, X. Zou, Chem. Sci. 2020, 11, 3397-3404). This would imply that the crystalline ZIF-8@NDV (E) composite may perform better in maintaining the stability of encapsulated vaccine. However, the significantly large loss of viral titre for ZIF-8@NDV(E) due to synthesis conditions makes it an untenable formulation (FIG. 14b).

### Effect of Biomimetic Mineralization and Post-Processing

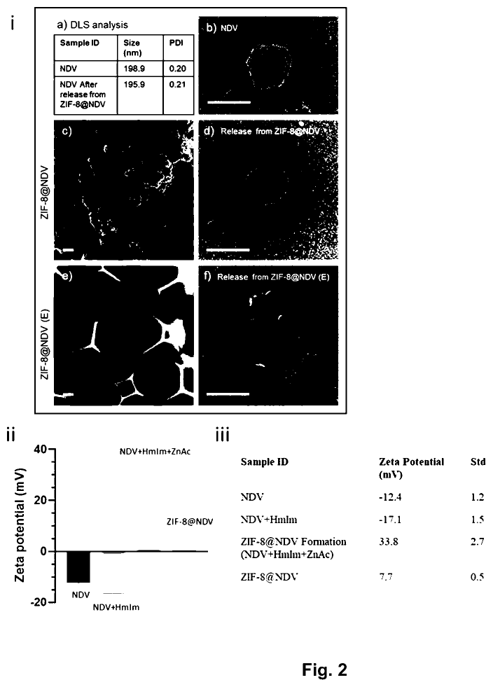

FIG. 2(a) Dynamic light scattering (DLS) analysis of the virus particles or virions before encapsulation and after their release from the ZIF-8@NDV show similar particle size indicating structural integrity is maintained during the encapsulation and release process. This was also confirmed using SEM and TEM. When the water-washed, amorphous ZIF-8@NDV composite in (c) was dissociated using the release buffer, the TEM analysis (using negative staining) demonstrates structurally intact virus particles. However, when the ZIF-8@NDV composite is post-processed using an ethanol solution to give rhombic decahedral ZIF-8@NDV(E) composite in (e), the virions released from this composite appeared to have lost their structural integrity as shown in the negative contrast TEM image (f) (scale—200 nm).

Surface charge assessment in FIG. 2(ii) Zeta potential plot of pristine NDV virus followed by virus with addition of first precursor (2-methylimidazole—Hmlm) and second precursor (Zinc Acetate ZnAc) and the final ZIF-8@NDV composites after the synthesis and washing steps. Y-axis units expressed as millivolt. iii) Zeta potential values as plotted in (i); error=standard deviation Std. (n=3).

The surface charge analysis shows the initial negative surface charge of the virions, NDV which is enhanced on addition of the imidazole precursor. The addition of the zinc salt inverts the zeta potential to nearly (+)34 mV. We postulate this is due to the presence of excess precursors as the final composite post-washing steps have a zeta potential between (+)7-8 mV.

The zeta potential measurements were used to investigate MOF growth around the negatively charged surface of NDV.

### Optimisation of ZIF-8@NDV Synthesis Parameters

ZIF-8@NDV composite was synthesised by adapting the method previously described by Kang et. al. (K. Liang, C. J. Coghlan, S. G. Bell, C. Doonan, P. Falcaro, Chemical communications 2016, 52, 473-476; K. Liang, R. Ricco, C. M. Doherty, M. J. Styles, S. Bell, N. Kirby, S. Mudie, D. Haylock, A. J. Hill, C. J. Doonan, Nature communications 2015, 6, 7240). Briefly, the addition of 100 μL of an aqueous solution of 2×‘X’ mM 2-methylimidazole (Hmlm) to 200 μL of NDV in allantoic fluid at a virus titre of ‘A’ TCID50/mL, followed by 2×′Y′ mM of zinc acetate (ZnAc) solution (aq.) resulted in immediate flocculation. The ZIF-8@NDV composite was left to precipitate for 30 min followed by centrifugation, washing and centrifugation to collect the ZIF-8@NDV pellet. For initial screening, the ‘X’ ‘Y’ and ‘A’ were varied as shown in FIG. 14a with decreasing virus titre ‘A’ in rows (downward) and increasing precursor concentration, X and Y in columns (left to right). Y:X is kept constant at 1:4 (precursor concentration ratio). The undiluted aliquot of NDV in allantoic fluid, ˜10¹² TCID50/mL with precursor concentration Hmlm:ZnAc above 160:40 (100 μL, 320 mM Hmlm+100 μL, 80 mM ZnAc+200 μL NDV) demonstrated the larger pellet sizes. At lower precursor concentrations, the presence of NDV triggers biomimetic mineralization of ZIF-8, as no flocculation and precipitation was observed in the absence of the virus. Considering lower precursor concentrations, at which the presence of NDV triggers biomimetic mineralization of ZIF-8, we tested ZIF-8@NDV synthesis using reaction conditions with Hmlm:ZnAc, of 160:40 and 320:80, respectively. The two composites were tested for their viral titer using the TCID50 assay as shown in FIG. 14b.

While ZIF-8@NDV (160:40) had a higher mean 2.44×10¹² TCID50/mL, ZIF-8@NDV (320:80) was not significantly different from the control, NDV (FIG. 14b). However, a significantly small residual titre in both the supernatants inferred good encapsulation efficiency. To confirm the ZIF-8 composition using XRD, a crystalline ZIF-8@NDV formulation using 50% ethanol post-processing was synthesised. The resulting ZIF-8@NDV (E) formulation had an anticipated loss of 8.2 log TCID50/mL of the NDV titre. The order of addition of precursors to the NDV in alantoic fluid for ZIF-8@NDV synthesis were the organic ligand solution, Hmlm (160 mM, pH 10.5) followed by the ZnAc (40 mM, pH 6.0) solution. The converse, ZIF-8@NDV (160:40) M2 yielded a composite with lower residual titre (10.67 log TCID50/mL). This could be due to the known high affinity of zinc ions to the viral capsid proteins and consequent loss of physical processes such as virus attachment, infection, uncoating and its replication potential (S. A. Read, S. Obeid, C. Ahlenstiel, G. Ahlenstiel, Advances in nutrition 2019, 10, 696-710; U. C. Chaturvedi, R. Shrivastava, FEMS Immunology & Medical Microbiology 2005, 43, 105-114; c) A. J. Te Velthuis, S. H. van den Worm, A. C. Sims, R. S. Baric, E. J. Snijder, M. J. van Hemert, PLOS pathogens 2010, 6, e1001176). However, in the presence of the organic ligand and later introduction of zinc ions favours the framework formation and resulting encapsulation of the live virions with an unaffected titre. (all values are means±SD, statistical significance tested using one-way ANOVA p<0.05 with Dunett's multiple comparison test).

SAXS spectra for pure ZIF-8, ZIF-8@NDV (E) (50% ethanol post-processing) were identical confirming their phase identity, while ZIF-8@NDV ((amorphous; aqueous synthesis) was an amorphous composite with no distinct peaks in its SAXS spectra (FIG. 15).

System Versatility: Encapsulation of Influenza A strain WSN in ZIF-8 and Aluminium Fumarate MOFs

The versatility of the biomimetic mineralization technique is demonstrated by its extension to another live virus, Influenza A WSN strain, forming a ZIF-8@WSN composite. The application of another MOF was also evaluated by encapsulating WSN in aluminium fumarate MOF to form Alfum@WSN. The TCID50/mL values indicate good encapsulation efficiency and recovery of WSN in both the ZIF-8 and Alfum MOFs.

All data is presented as mean±SD (n=3). Statistical significance was tested against the control (WSN) group (one-way ANOVA p<0.05 with Dunett's multiple comparison test; **, p<0.01, ****, p<0.0001).

Sodium citrate buffer (pH 5.0, 50 mM) was used to release the virus load from ZIF-8@WSN, while an EDTA solution (100 mM, pH 7.0) was used to chelate aluminium and release WSN from the AlFum@WSN composite.

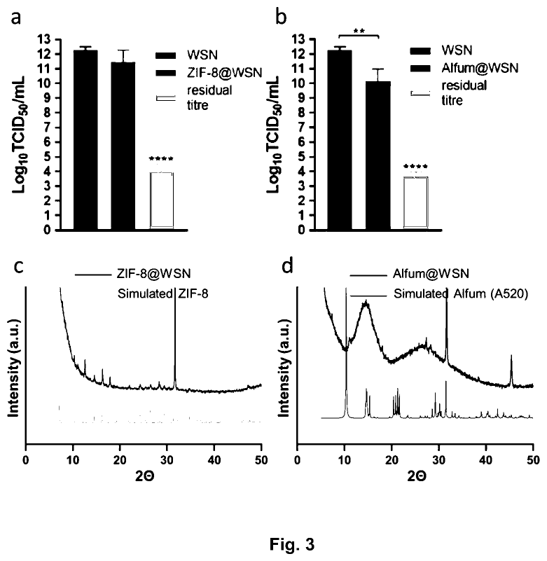

FIG. 3 a shows that ZIF-8@WSN demonstrated good encapsulation efficiency with no significant drop in titre. FIG. 3(c) shows simulated XRD for ZIF-8 and the ZIF-8@WSN composite, which had a minor trace crystalline and similar to ZIF-8@NDV (FIG. 1c) the majority of the composite is an amorphous structure. The successful encapsulation of WSN using Aluminium fumarate (Alfum) forming Alfum@WSN is also demonstrated. However, as shown in FIG. 3b, the Alfum biomimetic mineralization process has lower efficiency than ZIF-8 encapsulation, with significant reduction in viral titre (average 2.14 log TCID50/mL) decrease during the encapsulation and release steps. The order in which the precursors are added is an important determinant of the viability of the resulting Alfum@WSN composite. When an acidic solution of fumaric acid (˜pH 3.0) is added to WSN prior to sodium aluminate (˜pH 8-9), the resulting Alfum@WSN has a significant 9.15 log TCID50/mL loss of viral titre (FIG. 16b). This highlights that pH sensitivity and effect of ionic strength on viral integrity influences the synthesis conditions to adapt for its biomimetic encapsulation. A higher precursor concentration was also evaluated; however, it provides no advantage with a significantly lower viral titre relative to Alfum@WSN (FIG. 16b).

FIG. 3(d) Shows a simulated spectrum for Aluminium Fumarate MOF (Basolite A520) and Alfum@WSN. The water-based methodology led to the synthesis of an amorphous MOF encapsulating the viruses. In case of WSN, the higher (Hmlm/ZnAc; 320 mM/80 mM) precursor concentrations show about 1.8 log TCID50/mL higher viral titre (FIG. 16a).

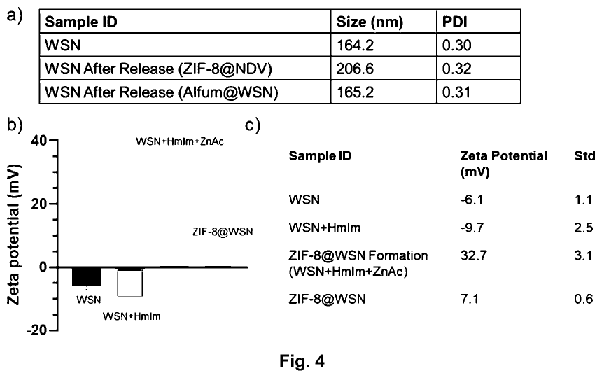

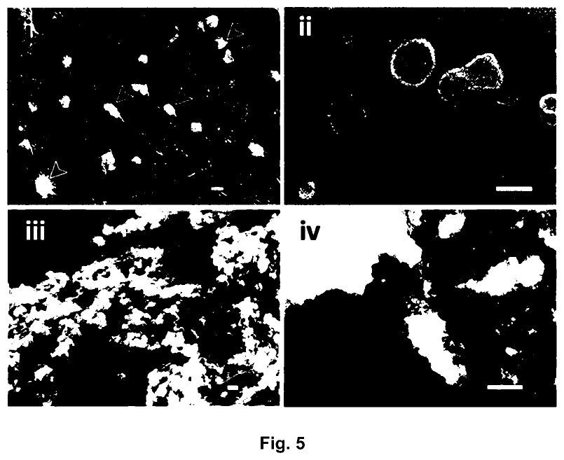

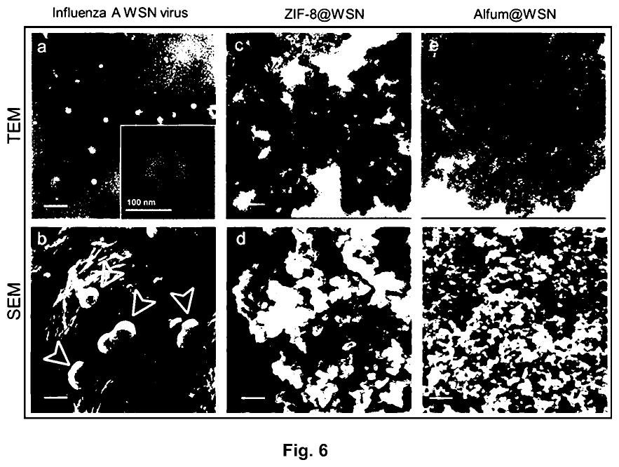

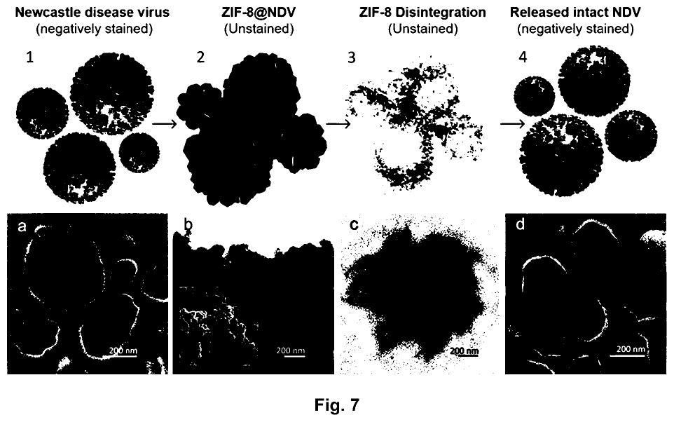

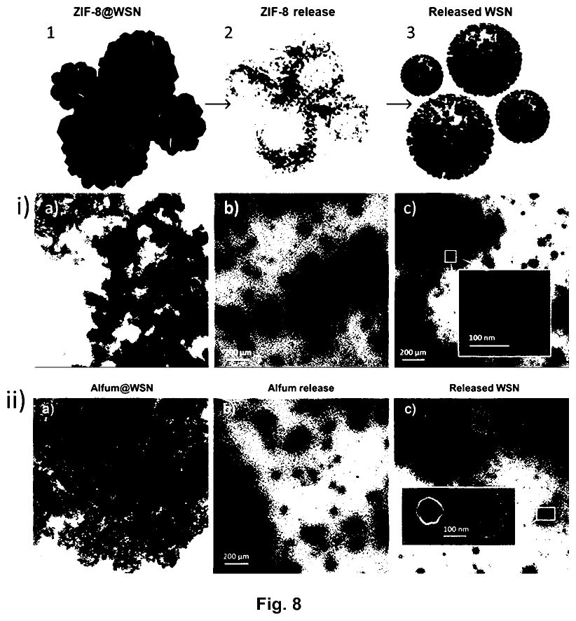

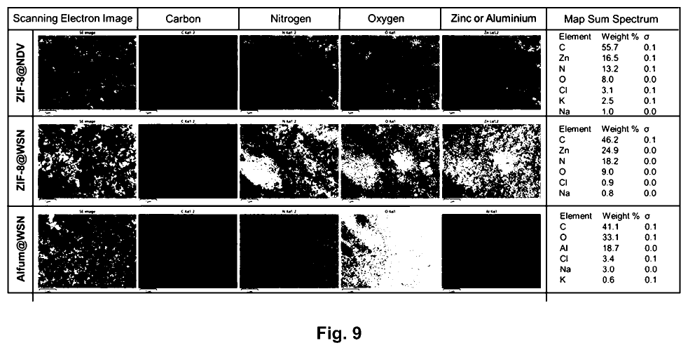

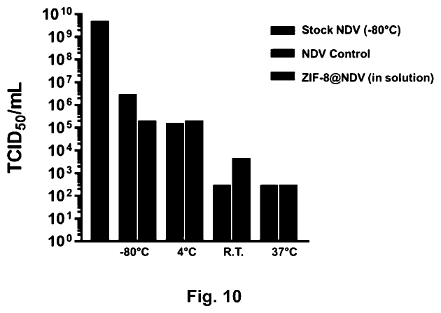

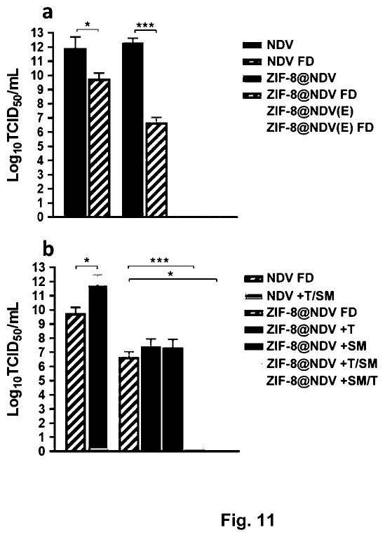

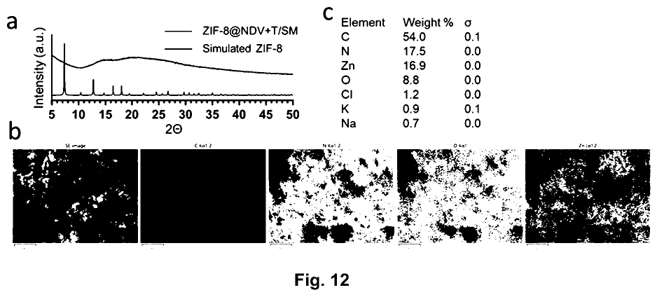

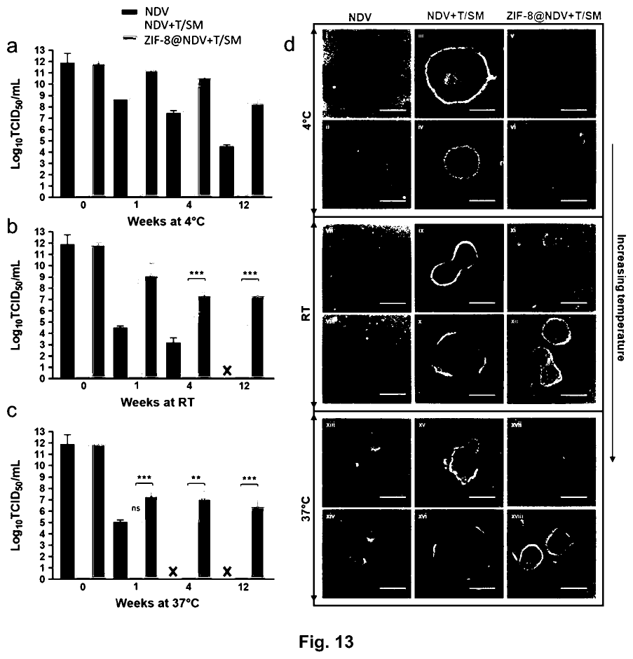

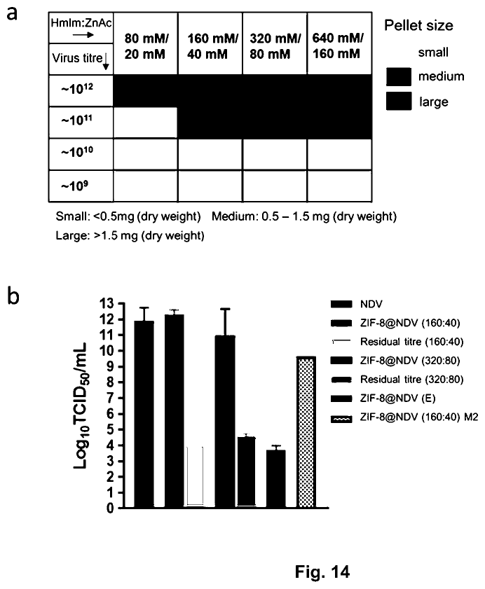

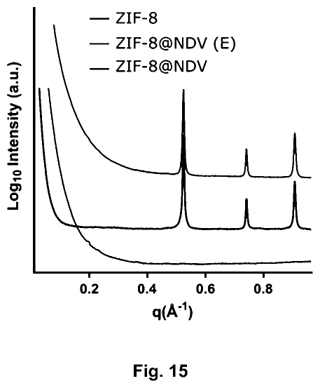

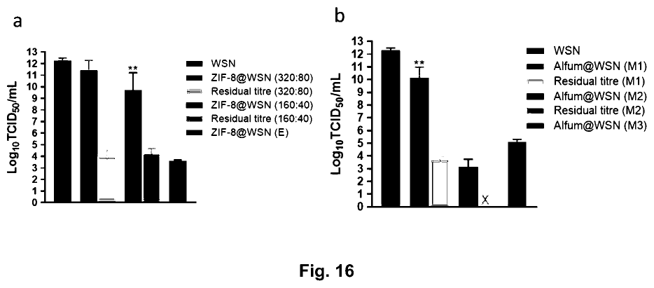

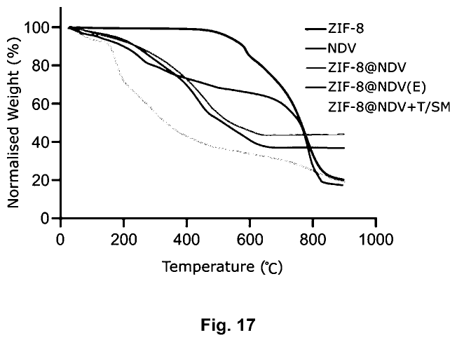

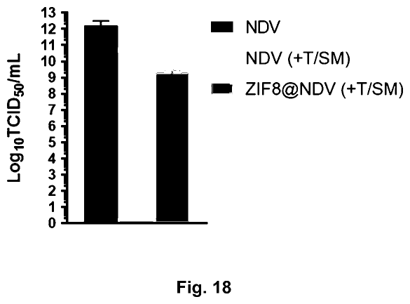

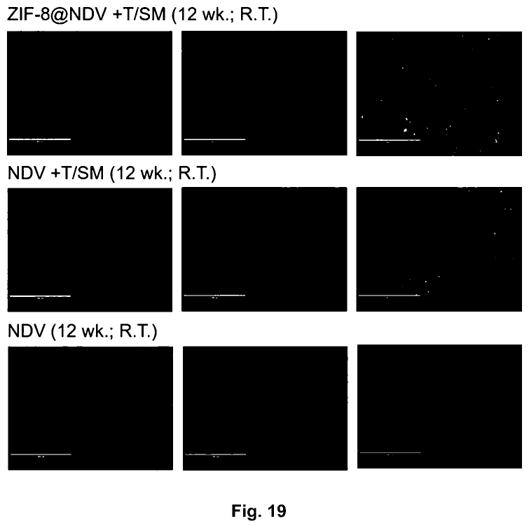

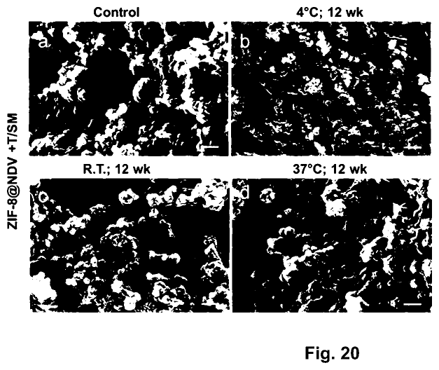

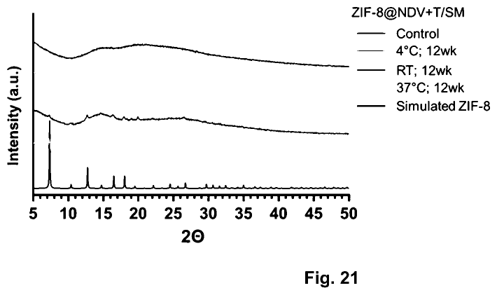

FIG. 4(a) shows nanoparticle size derived from Dynamic light scattering (DLS) analysis of the virus particles or virion size before encapsulation and after their release from the ZIF-8@WSN and Alfum@WSN. The results indicate that structural integrity is maintained during the encapsulation and release process.

FIG. 4(b) shows zeta potential plots of pristine WSN virus followed by the virus with addition of first precursor (2-methylimidazole—Hmlm) and second precursor (Zinc Acetate ZnAc) and the final ZIF-8@WSN composites after the synthesis and washing steps. Y-axis units expressed as millivolt. FIG. 4(c) are the zeta potential values as plotted in FIG. 4(b); error=standard deviation Std. (n=3). The surface charge analysis shows that the initial negative surface charge of the virions, WSN gets slightly enhanced on addition of the imidazole precursor. Following this, the addition of the zinc salt inverts the zeta potential to nearly (+)33 mV. The present inventors postulate this is due to the presence of excess precursors as the final composite post-washing steps have a zeta potential of about (+)7 mV.

FIG. 5 shows the scanning electron microscopy and transmission electron microscopy images for NDV (i, ii), and ZIF-8@NDV (iii, iv) (scale—200 nm).

FIGS. 6 (a) and (b) show the pristine WSN virus, inset in (a) is a higher magnification image of WSN while one can visualize the morphology in (b) pointed using yellow arrows. (c, d) show the TEM and SEM micrographs for the MOF encapsulated ZIF-8@WSN composite and (e,f) show TEM and SEM micrographs for the MOF encapsulated Alfum@WSN composite (scale—200 nm).

Due to its nanometre size resolution, transmission electron microscopy (TEM) is the only microscopic technique that allows direct visualization of viruses. The sequence of events using the biomimetic mineralization technique, from NDV encapsulation in the ZIF-8 MOF to its release using the citrate buffer prior to the TCID50 infection were imaged using the TEM. FIG. 7a shows the structure of NDV virions revealed by negative contrast using phosphotungstic acid stain. Virions ranging in size between 150 to 400 nm were seen with glycoproteins, haemagglutinin-neuraminidase and fusion (F) proteins apparent as tiny spikes projecting from the external surface of the membrane. The formation of ZIF-8@NDV generated an amorphous composite which appears like a material precipitate in the electron image FIG. 7b that correlates with the inset SEM image from the sample. As soon as the release buffer is added to the composite, the MOF disintegrates. The unstained image in FIG. 7c captures this incomplete dissolution process with some of the electron dense MOF material trace around the surface of virions. On complete dissolution, the negative contrast image FIG. 7d shows the structural integrity of the released virions which confirms that they remain intact after the encapsulation and release protocol.

FIG. 8 Schematic Steps 1 to 3 show the sequence of events captured using TEM images in (i), The WSN virus is encapsulated in the MOF forming an amorphous composite (a). A sodium citrate buffer (pH 5.0, 50 mM) was used to chelate the zinc ions causing MOF disintegration (b) releasing structurally intact WSN virions (c, inset c). Image b) and c) are both from the released sample, however, with the help of a negative contrast staining in c) one can visualize the virus while b) clearly shows the MOF disintegration. FIG. 8(ii) shows the WSN virus is encapsulated in the MOF forming Alfum@WSN composite (a). An EDTA solution (100 mM) was used to chelate the Aluminium ions causing MOF disintegration as shown in the TEM image (b) releasing structurally intact WSN virions (c, inset c). Image b) and c) are both from the released sample, however, with the help of a negative contrast staining in c) we can visualize the virus while b) clearly shows the MOF disintegration. Scale bars as marked.

FIG. 9 shows energy-dispersive x-ray spectroscopy (EDS) of ZIF-8@NDV, ZIF-8@WSN, and Alfum@WSN confirming the expected elements in a biological sample, C, H, O, N along with a homogeneous distribution of either zinc (green) or aluminium (royal blue) shows consistent and uniformly MOF mineralized composition of the vaccine formulations. (scale=1 μm). The elemental composition (weight %) for each sample is shown in the corresponding table on right (o=standard deviation).

The present inventors performed a preliminary test by leaving the wet pellet form of ZIF-8@NDV along with control NDV aliquots at 4 different temperature conditions, −80° ° C., 4° C., R.T. and 37° C. over a period of 3 weeks (FIG. 10). The initial drop in infectious titre (drop from black to grey at −80° C.) of stock NDV occurs due to it freeze-thaw cycle. The wet-pellet or solution form of the ZIF-8@NDV presented no significant advantage over the control, warranting the need to investigate lyophilization.

### ZIF-8@WSN and Alfum@WSN

Encapsulation efficiency and infectivity of ZIF-8@WSN and Alfum@WSN.

FIG. 16(a) shows the TCID50/mL results from ZIF-8@WSN infection assay, ZIF-8@WSN was synthesised using precursor concentrations resulting in Hmlm:ZnAc of 160:40 and 320:80 in the reaction solution. Unlike ZIF-8@NDV, for ZIF-8@WSN the 320:80, Hmlm:ZnAc concentration resulted in a higher infective titre of 8.1×10¹¹ TCID50/mL, not significantly different from the control WSN. In addition, a small residual titre in the supernatant indicates good encapsulation efficiency. On the other hand, using lower (160:40) precursor concentrations synthesised ZIF-8@WSN with a significant 2.6 log TCID50/mL loss of viral titre. Like for ZIF-8@NDV, in an attempt to synthesise a crystalline ZIF-8@WSN formulation using 50% ethanol post-processing, the product formulation ZIF-8@WSN (E) resulted in a significant loss of 8.7 log TCID50/mL of the WSN titre. (all values are means±SD, statistical significance tested using one-way ANOVA p<0.05 with Dunett's multiple comparison test).

FIG. 16(b) Encapsulation efficiency and infectivity of Alfum@WSN Alfum@WSN was synthesised using three methods, M1, M2, M3 with varying parameters (FIG. 16b).

Briefly, for M1, 300 μL of sodium aluminate solution (45 mM) was added to 300 μL of WSN in allantoic fluid followed by 300 μL of fumaric acid solution (45 mM); M2, the order of the addition of precursor solutions in M1 was reversed and in M3, to increase the ratio of MOF precursors to vaccine, 600 μL each of the precursor solutions were added to 300 UL of WSN in allantoic fluid. M1 yielded Alfum@WSN with the most viable titre, nevertheless it was 2.14 log TCID50/mL lower than the WSN control. M2 synthesised Alfum@WSN indicates WSN acid instability, resulting in an untenable formulation. Surprisingly, the increase in the amount of precursors also resulted in a lower titre, possibly due to spontaneous precipitation of Aluminium fumarate or decreased pH and poor encapsulation of the WSN. (all values are means±SD, statistical significance tested using one-way ANOVA p<0.05 with Dunett's multiple comparison test, **, p<0.001).

### Effect of Freeze Drying on Live Virus Vaccine and its MOF Composite

The ZIF-8@NDV, ZIF-8@WSN and Alfum@WSN formulations were used as synthesised in a wet pellet form for their encapsulation efficiency and virus titre quantification. However, live viral vaccines have limited storage stability in aqueous media especially above 8° C. (J. Peetermans, Factors affecting the stability of viral vaccines (In Developments in biological standardization) 1996, 87, 97-101). Therefore, the removal of bulk water (to 1-2%) content significantly improves their stability (L. Hansen, R. Daoussi, C. Vervaet, J.- P. Remon, T. De Beer, Vaccine 2015, 33, 5507-5519; D. Chen, D. Kristensen, Expert Rev. Vaccines 2009, 8, 547-557). With the exception of oral polio virus (J. Sokhey, C. K. Gupta, B. Sharma, H. Singh, Vaccine 1988, 6, 12-13), all live viral vaccines are freeze-dried (L. Hansen, R. Daoussi, C. Vervaet, J.- P. Remon, T. De Beer, Vaccine 2015, 33, 5507-5519). A dry powdered form is convenient to develop tablets, baits or other novel needle-free formulations. Freeze drying (FD) offers many advantages over other drying methods, including (i) The use of low temperatures for the drying process, (ii) Aseptic process for drying eliminating the need for additional sterilisation steps and (iii) The ease of reconstitution because of the resulting microporous formulations. However, the process of FD itself is associated with damage to the viral structure and its components including coat proteins and the lipid membrane by intra-virus ice formation, change in osmolarity, altered formulation buffer and other factors (L. Hansen, R. Daoussi, C. Vervaet, J.- P. Remon, T. De Beer, Vaccine 2015, 33, 5507-5519). FIG. 11a shows a significant (average 2.2 log TCID50/mL) decrease in NDV titre upon FD. The ZIF-8@NDV composite resulted in approximately 2.5 times greater (average 5.7 log TCID50/mL) decrease in its viral titre upon FD. The present inventors postulate that during the ZIF-8 encapsulation of NDV, constituents of the virus preparation medium such as salts and proteins that otherwise surround NDV and provide some protection from freeze-drying stresses are no longer present further destabilising the ZIF-8@NDV composite. The crystalline ZIF-8@NDV (E) composite is stable to FD, but regardless it cannot be applied due to an already low viral titre resulting from the synthesis conditions.

### Synthesis of a Stable Freeze-Dried MOF@Live Viral Vaccine Composite

To reduce the unfavourable impact of FD, the use of an optimised formulation and FD processes are preferred. One approach for optimization is to use a suitable combination of excipients that will have a specific function related to the process of drying and to the protection of the active component during and after FD. Several excipients including amino acids, sugars, sugar alcohols and proteins are used as cryoprotectants for several licensed live viral vaccines (L. Hansen, R. Daoussi, C. Vervaet, J.- P. Remon, T. De Beer, Vaccine 2015, 33, 5507-5519). The addition of excipients can be easily investigated with live viral vaccine but for a composite, the presence of the MOF adds complexity to the optimisation process. The viral quantification TCID50 assays are more time and resource extensive, than a simple and rapid spectrophotometric enzyme activity assay. The effect of sugars and proteins; bovine serum albumin (BSA), cellobiose, starch, skim milk, trehalose and sucrose have been investigated on stabilisation of a ZIF-8 encapsulated glucose oxidase (GOx) composite. The studies have shown complete protection of enzyme activity by a combination of (a) stabilizing the enzyme prior to ZIF-8 synthesis using 5% (w/w) skim milk (SM) and (b) the cryoprotection of the ZIF-8@GOx composite post-synthesis using 2% (w/w) trehalose (T). Therefore, the excipients T and SM were selected to investigate their effect to protect the ZIF-8@NDV viral titre during the FD process.

FIG. 11b shows an addition of 10% (w/v) trehalose or 10% (w/v) skim milk to NDV significantly enhance its infectivity by 2 log TCID50/mL in comparison with the NDV FD without any excipient protection. Trehalose and skim milk were added to the ZIF-8@NDV composite in combination before or after ZIF-8 synthesis. The addition of trehalose before ZIF-8 encapsulation of NDV for protecting the vaccine inside and of skim milk later to offer protection to the ZIF-8@NDV composite from the drying process (+T/SM), is an optimal combination providing a significant (average 3.76 log₁₀ TCID50/mL) increase in viral titre relative to ZIF-8@NDV FD. There was a relatively lower, yet a significant increase of 2 log TCID50/mL when the two excipients were incorporated vice versa (+SM/T).

The PXRD spectrum of ZIF-8@NDV+T/SM shows a predominantly amorphous phase similar to ZIF-8@NDV (FIG. 12). Energy-dispersive x-ray spectroscopy (EDS) of ZIF-8@NDV+T/SM a homogeneous distribution of either zinc (green) which shows consistent and uniformly MOF mineralized composition of the vaccine formulations. (scale=1 μm). The elemental composition (weight %) for each sample is shown in the corresponding table(c) (σ=standard deviation).

The thermogravimetric analysis (TGA) which stipulates mass loss rate as a factor of temperature was conducted from 100 to 900° C. (under N₂) at the rate of 10° C./min. The TGA plots indicate expedited decomposition of ZIF-8@NDV and ZIF-8@NDV(E), relative to the control ZIF-8 MOF. This is consistent with the presence of the live viral vaccine, NDV in the composite. Further, the addition of trehalose and skim milk led to a further drop in material stability for the ZIF-8@NDV+T/SM composite (FIG. 17).

Time and Temperature dependent stability of the MOF@Vaccine composite Aliquoted, lyophilised preparations of NDV; NDV+T/SM and ZIF-8@NDV+T/SM, and freshly thawed aliquots of NDV in allantoic fluid were placed at 4° C., room temperature (regulated at approximately 25° C.) and 37° C. for a period of 12 weeks. At regular time intervals, virus infectivity was measured by TCID50 assay on three samples (n=3) from each formulation type stored at each of the three temperature conditions, respectively.

The freeze dried ZIF-8@NDV (+T/SM) formulation was then investigated for its storage stability. The long-term storage stability of NDV, NDV(+T/SM) and ZIF-8@NDV(+T/SM) was evaluated for storage at 4° C., room temperature (RT) and 37° C., over a period of 1, 4 and 12 weeks, respectively. The initial titre for NDV (1.7×10¹² TCID50/mL) and NDV (+T/SM) (5.8×10¹⁰ TCID50/mL) were significantly higher than ZIF-8@NDV (+T/SM) (2.0×10⁹ TCID50/mL) as shown later in FIG. 18. For comparison, the titre for all three formulations is normalised to an initial starting virus titre of 1.7×10¹² TCID50/mL. Over a period of 1, 4 and 12 weeks at 4° C. (FIG. 13a), the NDV titre decreased by 3.3, 4.4 and 7.4 log TCID50/mL respectively. The titre at these time points were significantly lower than both the FD formulations, with and without ZIF-8 protection. Over the period of 12 weeks, the FD formulations, NDV (+T/SM) and ZIF-8@NDV (+T/SM) demonstrated a total decrease of 3 and 3.6 log TCID50/mL, respectively. Therefore, the loss in titre of the FD formulations in 12 weeks is comparable to the titre that the control NDV formulation lost within the very first week of storage at 4° C. The FD process significantly increased the stability of viruses by inhibiting the degradation and destabilization pathways that can occur in aqueous media; however, in these refrigerated conditions (4° C.), the ZIF-8 encapsulation does not impart any additional advantage to the FD NDV(+T/SM) formulation.

When exposed to higher room temperature conditions (FIG. 13b), the NDV titre declined rapidly demonstrating a significant loss of 7.4 and 8.7 log₁₀ TCID50/mL in the 1- and 4-week time period and furthermore a total loss of viral titre before 12 weeks. In these ambient conditions, even though the FD formulations, NDV (+T/SM) and ZIF-8@NDV (+T/SM) demonstrated significantly better protection of NDV titre, their stability was likewise affected resulting in nearly 3.3 and 7 log₁₀ TCID50/mL loss in NDV (+T/SM) titre over a period of 1 and 12 weeks, respectively. Interestingly, it was at this higher temperature, and at the longer storage durations in these conditions, the MOF encapsulated ZIF-8@NDV (+T/SM) formulation performed significantly better than the unprotected NDV(+T/SM) formulation with a mean 4.5 vs 6.4 and 4.5 vs 7 log₁₀ TCID50/mL loss in titre at 4 and 12 weeks, respectively.

Immunofluorescence images from DF1 cells infected with a 100× dilution of the original viral titre in form of ZIF-8@NDV+T/SM, NDV+T/SM and NDV (in Allantoic fuid) that were stored at room temperature for a period of 12 weeks (scale bar—400 μm) (FIG. 19).

Fluorescence images were captured using the EVOS FL Imaging system with high resolution CMOS camera. The Alexa-fluor488 labelled secondary antibody facilitated fluorescent green labelling for viral antigen and cell nuclei were labelled blue using DAPI stain.

Accelerated stability testing was performed at 37° C. and consequently, all the three formulations were rendered more labile (FIG. 13c). The control live viral vaccine, NDV was unsustainable in these conditions with total loss of titre by week 4 and the FD unprotected NDV(+T/SM) was not too different to the control with 6.7 log₁₀ TCID50/mL loss in titre in the first week. However, once again MOF encapsulation provided significant protection with a loss of 4.5 vs 6.7, 4.8 vs 6.7 and 5.4 vs 7.7 log₁₀ TCID50/mL at 1, 4 and 12 weeks of storage for the ZIF-8@NDV(+T/SM) and NDV(+T/SM), respectively. These data indicate that the excipient stabilized freeze-dried formulation of NDV markedly protects the NDV infectious titre. However, at the more demanding test conditions, which is beyond a threshold temperature condition and at longer storage durations, ZIF-8 encapsulation provides structural protection further imparting significant stabilisation to the NDV and NDV (+T/SM) FD formulations.

FIG. 13d provides a visualization of the degree of loss in structural integrity of NDV in each of the three test composites after the 12-week thermal treatment. The structure is revealed using TEM, by negative contrast (phosphotungstic acid) staining of the NDV virions from the NDV, NDV+T/SM and released virions from the ZIF-8@NDV(E) composite stored at 4° ° C., RT, and 37° C., for 12 weeks. The effect of thermal ageing on the ZIF-8@NDV+T/SM composite was analysed using SEM and

PXRD on the ZIF-8@NDV+T/SM control (t=0) and the composite after 12 weeks (t=12 wk) of storage at 4° C., RT, and 37° C., respectively (FIGS. 20 and 21). While the SEM micrographs do not indicate any distinct difference in the morphology of the composite, the PXRD data shows an increasing trace of the crystalline ZIF-8 phase esp. after storage at the highest temperature of 37° C. for 12 wk. The present inventors have previously observed similar phenomena in their in-situ SAXS thermal treatment of a ZIF biomimetic mineralized enzyme, which showed ZIF-8 crystalisation upon heat treatment around 45° C. (R. Singh, M. M. Musameh, Y. Gao, B. Ozcelik, and C. M. Doherty. “Stable MOF@ Enzyme Composites for Electrochemical Biosensing Devices.” J. Mater. Chem. C, 2021, 9, 7677-7688). Ongoing research is focussed on understanding the mechanism of MOF encapsulation mediated thermal stabilization which will help understand this phenomenon in greater detail.

### Discussion

Amidst the uncertainties of the present COVID-19 pandemic, the deployment of the approved vaccine candidates has become the topmost global priority. Whilst the worldwide race to finding an effective vaccine was intense with many promising outcomes, it seems to be only half the battle won. The challenge of vaccine distribution across the world has reminded us that the elimination of vaccine ‘cold-chain’ logistics is the ‘holy grail’ of widespread immunization. This work demonstrates the first MOF encapsulated live viral vaccine formulations. The biomimetic mineralization of ZIF-8 MOF on NDV strain V4 live viral vaccine and ZIF-8 and Aluminium fumarate MOFs on Influenza A WSN viral strain, forming the ZIF-8@NDV, ZIF-8@WSN and AlFum@WSN formulations, respectively is demonstrated. It was confirmed that during the MOF encapsulation and the subsequent release processes the virus maintains its structural integrity demonstrated by the TEM imaging and it retains its functional integrity demonstrated by retention of viral replication potential in vitro. The MOF@vaccine formulations have a facile preparation in ambient aqueous conditions using a simple, rapid and scalable approach with cost-effective ingredients. Working with two different viruses and two diverse MOFs we highlighted the versatility of the technique and specific synthetic conditions required for each system. FD is the preferred method for live viral vaccine stabilization; however, FD stresses were detrimental to the NDV and even more to the ZIF-@NDV titre. Therefore, for further studies a potent FD formulation, ZIF-8@NDV+T/SM was developed using an optimum combination of trehalose and skim milk as stabilizing excipients. ZIF-8@NDV+T/SM, NDV+T/SM and NDV were compared for their storage stability at 4° C., room temperature and 37° C. over a period of 12 weeks. The application of stabilizing excipients and freeze-drying provides stability to the live viral vaccine at 4° C. which is consistent with the fact that most FD vaccine formulations still need to be refrigerated. Importantly, ZIF-8 MOF encapsulation significantly stabilized the vaccine in ambient conditions beyond the 4-week time point and at all time points at the higher temperature of 37° C.

1. A stabilized composition comprising a live viral vaccine, a live-attenuated viral vaccine, an inactivated viral vaccine, a chimeric viral vaccine, a virus like particle vaccine, or a viral vector encapsulated within a Metal Organic Framework (MOF) shell.

2. The stabilized composition of claim 1, wherein the live-attenuated or inactivated viral vaccine is a whole pathogen live-attenuated or inactivated viral vaccine

3. The stabilized composition of claim 1 or claim 2, wherein the vaccine or vector is replication competent.

4. The stabilized composition of any one of claims 1 to 3, wherein the composition is characterized as having improved stability over 12 weeks as compared to a comparative composition comprising the vector or vaccine without the MOF shell.

5. The stabilized composition of any one of claims 1 to 4, wherein the composition comprises at least 1 log₁₀ more virus after 12 weeks of storage at temperatures up to 37° C. as compared to a comparative composition comprising the vector or vaccine without the MOF shell.

6. The stabilized composition of any one of claims 1 to 5, wherein the composition maintains at least 5 log₁₀ of its viral replication potential or infectivity potential or its ability to deliver an antigenic payload after 12 weeks of storage at temperatures up to 37° C.

7. The stabilized composition of any one of claims 1 to 6, wherein the MOF is a zeolitic imidazolate framework (ZIF).

8. The stabilized composition of claim 7, wherein the ZIF is ZIF-8, ZIF-10, ZIF-90 or, ZIF-L.

9. The stabilized composition of claim 7 or claim 8, wherein the ZIF is ZIF-8.

10. The stabilized composition of any one of claims 1 to 6, wherein the MOF is aluminium fumarate.

11. The stabilized composition of any one of claims 1 to 10, wherein the composition is an amorphous composite?

12. The stabilized composition of any one of claims 1 to 11, wherein the composition is dried.

13. The stabilized composition of any one of claims 1 to 12, wherein the composition comprises one or more excipients.

14. The stabilized composition of claim 13, wherein the composition comprises, trehalose, or skim milk, or a combination thereof.

15. A method for producing a stabilized composition, the method comprising:
a. providing a live viral vaccine, a live-attenuated viral vaccine, an inactivated viral vaccine, a chimeric viral vaccine, a virus like particle vaccine, or a viral vector;
b. providing a ligand precursor;
c. providing a metal salt;
d. reacting the vaccine or vector, the ligand precursor and the metal salt to form a metal organic framework shell encapsulating the vaccine or vector.

16. The method of claim 15, wherein one or more of the vaccine or vector, the ligand precursor and the metal salt are provided in solution in one or mixed polar solvents.

17. The method of claim 16, wherein the solvent is water, alcohol, or other organic solvent.

18. The method of claim 16 or claim 17, wherein the solution comprises one or more excipients.

19. The method of any one of claims 15 to 18, wherein the ligand precursor is 2-methylimidazole.

20. The method of any one of claims 15 to 19, wherein the ligand precursor is 80 to 640 mM 2-methylimidazole in water.

21. The method of any one of claims 15 to 20, wherein the metal salt is zinc acetate.

22. The method of any one of claims 15 to 21, wherein the metal salt is 20 to 160 mM zinc acetate dihydrate in water.

23. The method of any one of claims 15 to 22, wherein the metal salt:ligand precursor ratio is between 1:4 and 1:8 mM.

24. The method of any one of claims 15 to 18, wherein the ligand precursor is sodium aluminate.

25. The method of any one of claim 15 to 18, or 24, wherein the ligand precursor is 5 to 45 mM sodium aluminate in water.

26. The method of any one of claims 15 to 18, or 24 and 25, wherein the metal salt is fumaric acid.

27. The method of any one of claim 15 to 18, or 24 to 26, wherein the metal salt is 5 to 45 mM fumaric acid in water.

28. The method of any one of claims 15 to 18 and 24 to 27, wherein the metal salt:ligand precursor ratio is 1:1.

29. The method of any one of claims 15 to 28, wherein the vaccine or vector, the ligand precursor and the metal salt solution are incubated for about 30 minutes.

30. The method of any one of claims 15 to 29, wherein the method further comprises centrifuging the reaction mixture of step (d) to pellet the metal organic framework encapsulating the vaccine or vector.

31. The method of any one of claims 15 to 30, wherein the method further comprises adding one or more excipients before the metal organic framework shell forms.

32. The method of claim 31, wherein the excipient is trehalose.

33. The method of any one of claims 30 to 32, wherein the pellet is collected.

34. The method of any one of claims 30 to 33, wherein the pellet is dried.

35. The method of claim 34, wherein the method further comprises adding one or more excipients prior to drying.

36. The method of claim 35, wherein the excipient is skim milk.

37. A method of preparation of the vaccine composition of any one of claims 1 to 14 for administration, wherein the method comprises adding a release buffer to the vaccine composition to chelate the metal ions causing MOF disintegration, and thereby release the vaccine or vector.

38. The method of claim 37, wherein the release buffer is sodium citrate.
